SEQUENCE LISTING (174 sequences)

SEQ ID NO: 1 - protein, 80 aa, Cryptomeria japonica
MKVTVAFNQFGPNRRVFIKRVSNVIIHGRRIDIFASKNFHLQKNTIGTGRRISLKLTSGKIASRRVDGIIAAYQNPASWK

SEQ ID NO: 2 - protein, 105 aa, Cryptomeria japonica
MKVTVAFNQFGPNRRVFIKRVSNVIIHGRRIDIFASKNFHLQKNTIGTGRRWKNNRIWLQFAKLTGFTLMGRRLKMPMYIAGYKTFDGRRVDGIIAAYQNPASWK

SEQ ID NO: 3 - protein, 134 aa, Cryptomeria japonica
MKVTVAFNQFGPNRRVFIKRVSNVIIHGRRIDIFASKNFHLQKNTIGTGRRWKNNRIWLQFAKLTGFTLMGRRPLWIIFSGNMNIKLKMPMYIAGYKTFDGRRAEVSYVHVNGAKFIRRVDGIIAAYQNPASWK

SEQ ID NO: 4 - protein, 31 aa, Cryptomeria japonica
IFSKNLNIKLNMPLYIAGNKRRFIKRVSNVI

SEQ ID NO: 5 - protein, 31 aa, Cryptomeria japonica
SSGKNEGTNIYNNNEAFKVERRFIKRVSNVI

SEQ ID NO: 6 - protein, 12 aa, Cryptomeria japonica
ISLKLTSGKIAS

SEQ ID NO: 7 - protein, 9 aa, Cryptomeria japonica
FIKRVSNVI

SEQ ID NO: 8 - protein, 17 aa, Cryptomeria japonica
EEYLILSARDVLAVVSK

SEQ ID NO: 9 - protein, 15 aa, Cryptomeria japonica
KSMKVTVAFNQFGPN

SEQ ID NO: 10 - protein, 32 aa, Cryptomeria japonica
EEYLILSARDVLAVVSKRRMKVTVAFNQFGPN

SEQ ID NO: 11 - protein, 20 aa, Cryptomeria japonica
IFSKNLNIKLNMPLYIAGNK

SEQ ID NO: 12 - protein, 20 aa, Cryptomeria japonica
SSGKNEGTNIYNNNEAFKVE

SEQ ID NO: 13 - protein, 13 aa, Cryptomeria japonica
VFIKRVSNVIIHG

SEQ ID NO: 14 - protein, 9 aa, Cryptomeria japonica
SIKRVSNVI

SEQ ID NO: 15 - protein, 15 aa, Cryptomeria japonica
  MISC_FEATURE (1)..(15): Cryj1 peptide, Figure 1, Row 1
DNPIDSCWRGDSNWA

SEQ ID NO: 16 - protein, 15 aa, Cryptomeria japonica
  MISC_FEATURE (1)..(15): Cryj1 peptide, Figure 1, Row 2
SCWRGDSNWAQNRMK

SEQ ID NO: 17 - protein, 15 aa, Cryptomeria japonica
  MISC_FEATURE (1)..(15): Cryj1 peptide, Figure 1, Row 3
DSNWAQNRMKLADCA

SEQ ID NO: 18 - protein, 15 aa, Cryptomeria japonica
  MISC_FEATURE (1)..(15): Cryj1 peptide, Figure 1, Row 4
QNRMKLADCAVGFGS

SEQ ID NO: 19 - protein, 15 aa, Cryptomeria japonica
  MISC_FEATURE (1)..(15): Cryj1 peptide, Figure 1, Row 5
LADCAVGFGSSTMGG

SEQ ID NO: 20 - protein, 15 aa, Cryptomeria japonica
  MISC_FEATURE (1)..(15): Cryj1 peptide, Figure 1, Row 6
VGFGSSTMGGKGGDL

SEQ ID NO: 21 - protein, 15 aa, Cryptomeria japonica
  MISC_FEATURE (1)..(15): Cryj1 peptide, Figure 1, Row 7
STMGGKGGDLYTVTN

SEQ ID NO: 22 - protein, 15 aa, Cryptomeria japonica
  MISC_FEATURE (1)..(15): Cryj1 peptide, Figure 1, Row 8
KGGDLYTVTNSDDDP

SEQ ID NO: 23 - protein, 15 aa, Cryptomeria japonica
  MISC_FEATURE (1)..(15): Cryj1 peptide, Figure 1, Row 9
YTVTNSDDDPVNPAP

SEQ ID NO: 24 - protein, 15 aa, Cryptomeria japonica
  MISC_FEATURE (1)..(15): Cryj1 peptide, Figure 1, Row 10
SDDDPVNPAPGTLRY

SEQ ID NO: 25 - protein, 15 aa, Cryptomeria japonica
  MISC_FEATURE (1)..(15): Cryj1 peptide, Figure 1, Row 11
VNPAPGTLRYGATRD

SEQ ID NO: 26 - protein, 15 aa, Cryptomeria japonica
  MISC_FEATURE (1)..(15): Cryj1 peptide, Figure 1, Row 12
GTLRYGATRDRPLWI

SEQ ID NO: 27 - protein, 15 aa, Cryptomeria japonica
  MISC_FEATURE (1)..(15): Cryj1 peptide, Figure 1, Row 13
GATRDRPLWIIFSGN

SEQ ID NO: 28 - protein, 15 aa, Cryptomeria japonica
  MISC_FEATURE (1)..(15): Cryj1 peptide, Figure 1, Row 14
RPLWIIFSGNMNIKL

SEQ ID NO: 29 - protein, 15 aa, Cryptomeria japonica
  MISC_FEATURE (1)..(15): Cryj1 peptide, Figure 1, Row 15
IFSGNMNIKLKMPMY

SEQ ID NO: 30 - protein, 15 aa, Cryptomeria japonica
  MISC_FEATURE (1)..(15): Cryj1 peptide, Figure 1, Row 16
MNIKLKMPMYIAGYK

SEQ ID NO: 31 - protein, 15 aa, Cryptomeria japonica
  MISC_FEATURE (1)..(15): Cryj1 peptide, Figure 1, Row 17
KMPMYIAGYKTFDGR

SEQ ID NO: 32 - protein, 15 aa, Cryptomeria japonica
  MISC_FEATURE (1)..(15): Cryj1 peptide, Figure 1, Row 18
IAGYKTFDGRGAQVY

SEQ ID NO: 33 - protein, 15 aa, Cryptomeria japonica
  MISC_FEATURE (1)..(15): Cryj1 peptide, Figure 1, Row 19
TFDGRGAQVYIGNGG

SEQ ID NO: 34 - protein, 15 aa, Cryptomeria japonica
  MISC_FEATURE (1)..(15): Cryj1 peptide, Figure 1, Row 120
GAQVYIGNGGPCVFI

SEQ ID NO: 35 - protein, 15 aa, Cryptomeria japonica
  MISC_FEATURE (1)..(15): Cryj1 peptide, Figure 1, Row 21
IGNGGPCVFIKRVSN

SEQ ID NO: 36 - protein, 15 aa, Cryptomeria japonica
  MISC_FEATURE (1)..(15): Cryj1 peptide, Figure 1, Row 22
PCVFIKRVSNVIIHG

SEQ ID NO: 37 - protein, 15 aa, Cryptomeria japonica
  MISC_FEATURE (1)..(15): Cryj1 peptide, Figure 1, Row 23
KRVSNVIIHGLHLYG

SEQ ID NO: 38 - protein, 15 aa, Cryptomeria japonica
  MISC_FEATURE (1)..(15): Cryj1 peptide, Figure 1, Row 24
VIIHGLHLYGCSTSV

SEQ ID NO: 39 - protein, 15 aa, Cryptomeria japonica
  MISC_FEATURE (1)..(15): Cryj1 peptide, Figure 1, Row 25
LHLYGCSTSVLGNVL

SEQ ID NO: 40 - protein, 15 aa, Cryptomeria japonica
  MISC_FEATURE (1)..(15): Cryj1 peptide, Figure 1, Row 26
CSTSVLGNVLINESF

SEQ ID NO: 41 - protein, 15 aa, Cryptomeria japonica
  MISC_FEATURE (1)..(15): Cryj1 peptide, Figure 1, Row 27
LGNVLINESFGVEPV

SEQ ID NO: 42 - protein, 15 aa, Cryptomeria japonica
  MISC_FEATURE (1)..(15): Cryj1 peptide, Figure 1, Row 28
INESFGVEPVHPQDG

SEQ ID NO: 43 - protein, 15 aa, Cryptomeria japonica
  MISC_FEATURE (1)..(15): Cryj1 peptide, Figure 1, Row 29
GVEPVHPQDGDALTL

SEQ ID NO: 44 - protein, 15 aa, Cryptomeria japonica
  MISC_FEATURE (1)..(15): Cryj1 peptide, Figure 1, Row 30
HPQDGDALTLRTATN

SEQ ID NO: 45 - protein, 15 aa, Cryptomeria japonica
  MISC_FEATURE (1)..(15): Cryj1 peptide, Figure 1, Row 31
DALTLRTATNIWIDH

SEQ ID NO: 46 - protein, 15 aa, Cryptomeria japonica
  MISC_FEATURE (1)..(15): Cryj1 peptide, Figure 1, Row 32
RTATNIWIDHNSFSN

SEQ ID NO: 47 - protein, 15 aa, Cryptomeria japonica
  MISC_FEATURE (1)..(15): Cryj1 peptide, Figure 1, Row 33
IWIDHNSFSNSSDGL

SEQ ID NO: 48 - protein, 15 aa, Cryptomeria japonica
  MISC_FEATURE (1)..(15): Cryj1 peptide, Figure 1, Row 34
NSFSNSSDGLVDVTL

SEQ ID NO: 49 - protein, 15 aa, Cryptomeria japonica
  MISC_FEATURE (1)..(15): Cryj1 peptide, Figure 1, Row 35
SSDGLVDVTLSSTGV

SEQ ID NO: 50 - protein, 15 aa, Cryptomeria japonica
  MISC_FEATURE (1)..(15): Cryj1 peptide, Figure 1, Row 36
VDVTLSSTGVTISNN

SEQ ID NO: 51 - protein, 15 aa, Cryptomeria japonica
  MISC_FEATURE (1)..(15): Cryj1 peptide, Figure 1, Row 37
SSTGVTISNNLFFNH

SEQ ID NO: 52 - protein, 15 aa, Cryptomeria japonica
  MISC_FEATURE (1)..(15): Cryj1 peptide, Figure 1, Row 38
TISNNLFFNHHKVML

SEQ ID NO: 53 - protein, 15 aa, Cryptomeria japonica
  MISC_FEATURE (1)..(15): Cryj1 peptide, Figure 1, Row 39
LFFNHHKVMLLGHDD

SEQ ID NO: 54 - protein, 15 aa, Cryptomeria japonica
  MISC_FEATURE (1)..(15): Cryj1 peptide, Figure 1, Row 40
HKVMLLGHDDAYSDD

SEQ ID NO: 55 - protein, 15 aa, Cryptomeria japonica
  MISC_FEATURE (1)..(15): Cryj1 peptide, Figure 1, Row 41
LGHDDAYSDDKSMKV

SEQ ID NO: 56 - protein, 15 aa, Cryptomeria japonica
  MISC_FEATURE (1)..(15): Cryj1 peptide, Figure 1, Row 42
AYSDDKSMKVTVAFN

SEQ ID NO: 57 - protein, 15 aa, Cryptomeria japonica
  MISC_FEATURE (1)..(15): Cryj1 peptide, Figure 1, Row 43
KSMKVTVAFNQFGPN

SEQ ID NO: 58 - protein, 15 aa, Cryptomeria japonica
  MISC_FEATURE (1)..(15): Cryj1 peptide, Figure 1, Row 44
TVAFNQFGPNCGQRM

SEQ ID NO: 59 - protein, 15 aa, Cryptomeria japonica
  MISC_FEATURE (1)..(15): Cryj1 peptide, Figure 1, Row 45
QFGPNCGQRMPRARY

SEQ ID NO: 60 - protein, 15 aa, Cryptomeria japonica
  MISC_FEATURE (1)..(15): Cryj1 peptide, Figure 1, Row 46
CGQRMPRARYGLVHV

SEQ ID NO: 61 - protein, 15 aa, Cryptomeria japonica
  MISC_FEATURE (1)..(15): Cryj1 peptide, Figure 1, Row 47
PRARYGLVHVANNNY

SEQ ID NO: 62 - protein, 15 aa, Cryptomeria japonica
  MISC_FEATURE (1)..(15): Cryj1 peptide, Figure 1, Row 48
GLVHVANNNYDPWTI

SEQ ID NO: 63 - protein, 15 aa, Cryptomeria japonica
  MISC_FEATURE (1)..(15): Cryj1 peptide, Figure 1, Row 49
ANNNYDPWTIYAIGG

SEQ ID NO: 64 - protein, 15 aa, Cryptomeria japonica
  MISC_FEATURE (1)..(15): Cryj1 peptide, Figure 1, Row 50
DPWTIYAIGGSSNPT

SEQ ID NO: 65 - protein, 15 aa, Cryptomeria japonica
  MISC_FEATURE (1)..(15): Cryj1 peptide, Figure 1, Row 51
YAIGGSSNPTILSEG

SEQ ID NO: 66 - protein, 15 aa, Cryptomeria japonica
  MISC_FEATURE (1)..(15): Cryj1 peptide, Figure 1, Row 52
SSNPTILSEGNSFTA

SEQ ID NO: 67 - protein, 15 aa, Cryptomeria japonica
  MISC_FEATURE (1)..(15): Cryj1 peptide, Figure 1, Row 53
ILSEGNSFTAPNESY

SEQ ID NO: 68 - protein, 15 aa, Cryptomeria japonica
  MISC_FEATURE (1)..(15): Cryj1 peptide, Figure 1, Row 54
NSFTAPNESYKKQVT

SEQ ID NO: 69 - protein, 15 aa, Cryptomeria japonica
  MISC_FEATURE (1)..(15): Cryj1 peptide, Figure 1, Row 55
PNESYKKQVTIRIGC

SEQ ID NO: 70 - protein, 15 aa, Cryptomeria japonica
  MISC_FEATURE (1)..(15): 56
KKQVTIRIGCKTSSS

SEQ ID NO: 71 - protein, 15 aa, Cryptomeria japonica
  MISC_FEATURE (1)..(15): Cryj1 peptide, Figure 1, Row 57
IRIGCKTSSSCSNWV

SEQ ID NO: 72 - protein, 15 aa, Cryptomeria japonica
  MISC_FEATURE (1)..(15): Cryj1 peptide, Figure 1, Row 58
KTSSSCSNWVWQSTQ

SEQ ID NO: 73 - protein, 15 aa, Cryptomeria japonica
  MISC_FEATURE (1)..(15): Cryj1 peptide, Figure 1, Row 59
CSNWVWQSTQDVFYN

SEQ ID NO: 74 - protein, 15 aa, Cryptomeria japonica
  MISC_FEATURE (1)..(15): Cryj1 peptide, Figure 1, Row 60
WQSTQDVFYNGAYFV

SEQ ID NO: 75 - protein, 15 aa, Cryptomeria japonica
  MISC_FEATURE (1)..(15): Cryj1 peptide, Figure 1, Row 61
DVFYNGAYFVSSGKY

SEQ ID NO: 76 - protein, 15 aa, Cryptomeria japonica
  MISC_FEATURE (1)..(15): Cryj1 peptide, Figure 1, Row 62
GAYFVSSGKYEGGNI

SEQ ID NO: 77 - protein, 15 aa, Cryptomeria japonica
  MISC_FEATURE (1)..(15): Cryj1 peptide, Figure 1, Row 63
SSGKYEGGNIYTKKE

SEQ ID NO: 78 - protein, 15 aa, Cryptomeria japonica
  MISC_FEATURE (1)..(15): Cryj1 peptide, Figure 1, Row 64
EGGNIYTKKEAFNVE

SEQ ID NO: 79 - protein, 15 aa, Cryptomeria japonica
  MISC_FEATURE (1)..(15): Cryj1 peptide, Figure 1, Row 65
YTKKEAFNVENGNAT

SEQ ID NO: 80 - protein, 15 aa, Cryptomeria japonica
  MISC_FEATURE (1)..(15): Cryj1 peptide, Figure 1, Row 66
AFNVENGNATPQLTK

SEQ ID NO: 81 - protein, 15 aa, Cryptomeria japonica
  MISC_FEATURE (1)..(15): Cryj1 peptide, Figure 1, Row 67
NGNATPQLTKNAGVL

SEQ ID NO: 82 - protein, 15 aa, Cryptomeria japonica
  MISC_FEATURE (1)..(15): Cryj1 peptide, Figure 1, Row 68
PQLTKNAGVLTCSLS

SEQ ID NO: 83 - protein, 15 aa, Cryptomeria japonica
  MISC_FEATURE (1)..(15): Cryj1 peptide, Figure 1, Row 69
TKNAGVLTCSLSKRC

SEQ ID NO: 84 - protein, 15 aa, Cryptomeria japonica
  MISC_FEATURE (1)..(15): Cryj2 peptide, Figure 2, Row 1
AINIFNVEKYGAVGD

SEQ ID NO: 85 - protein, 15 aa, Cryptomeria japonica
  MISC_FEATURE (1)..(15): Cryj2 peptide, Figure 2, Row 2
NVEKYGAVGDGKHDC

SEQ ID NO: 86 - protein, 15 aa, Cryptomeria japonica
  MISC_FEATURE (1)..(15): Cryj2 peptide, Figure 2, Row 3
GAVGDGKHDCTEAFS

SEQ ID NO: 87 - protein, 15 aa, Cryptomeria japonica
  MISC_FEATURE (1)..(15): Cryj2 peptide, Figure 2, Row 4
GKHDCTEAFSTAWQA

SEQ ID NO: 88 - protein, 15 aa, Cryptomeria japonica
  MISC_FEATURE (1)..(15): Cryj2 peptide, Figure 2, Row 5
TEAFSTAWQAACKNP

SEQ ID NO: 89 - protein, 15 aa, Cryptomeria japonica
  MISC_FEATURE (1)..(15): Cryj2 peptide, Figure 2, Row 6
TAWQAACKNPSAMLL

SEQ ID NO: 90 - protein, 15 aa, Cryptomeria japonica
  MISC_FEATURE (1)..(15): Cryj2 peptide, Figure 2, Row 7
ACKNPSAMLLVPGSK

SEQ ID NO: 91 - protein, 15 aa, Cryptomeria japonica
  MISC_FEATURE (1)..(15): Cryj2 peptide, Figure 2, Row 8
SAMLLVPGSKKFVVN

SEQ ID NO: 92 - protein, 15 aa, Cryptomeria japonica
  MISC_FEATURE (1)..(15): Cryj2 peptide, Figure 2, Row 9
VPGSKKFVVNNLFFN

SEQ ID NO: 93 - protein, 15 aa, Cryptomeria japonica
  MISC_FEATURE (1)..(15): Cryj2 peptide, Figure 2, Row 10
KFVVNNLFFNGPCQP

SEQ ID NO: 94 - protein, 15 aa, Cryptomeria japonica
  MISC_FEATURE (1)..(15): Cryj2 peptide, Figure 2, Row 11
NLFFNGPCQPHFTFK

SEQ ID NO: 95 - protein, 15 aa, Cryptomeria japonica
  MISC_FEATURE (1)..(15): Cryj2 peptide, Figure 2, Row 12
GPCQPHFTFKVDGII

SEQ ID NO: 96 - protein, 15 aa, Cryptomeria japonica
  MISC_FEATURE (1)..(15): Cryj2 peptide, Figure 2, Row 13
HFTFKVDGIIAAYQN

SEQ ID NO: 97 - protein, 15 aa, Cryptomeria japonica
  MISC_FEATURE (1)..(15): Cryj2 peptide, Figure 2, Row 14
VDGIIAAYQNPASWK

SEQ ID NO: 98 - protein, 15 aa, Cryptomeria japonica
  MISC_FEATURE (1)..(15): Cryj2 peptide, Figure 2, Row 15
AAYQNPASWKNNRIW

SEQ ID NO: 99 - protein, 15 aa, Cryptomeria japonica
  MISC_FEATURE (1)..(15): Cryj2 peptide, Figure 2, Row 16
PASWKNNRIWLQFAK

SEQ ID NO: 100 - protein, 15 aa, Cryptomeria japonica
  MISC_FEATURE (1)..(15): Cryj2 peptide, Figure 2, Row 17
NNRIWLQFAKLTGFT

SEQ ID NO: 101 - protein, 15 aa, Cryptomeria japonica
  MISC_FEATURE (1)..(15): Cryj2 peptide, Figure 2, Row 18
LQFAKLTGFTLMGKG

SEQ ID NO: 102 - protein, 15 aa, Cryptomeria japonica
  MISC_FEATURE (1)..(15): Cryj2 peptide, Figure 2, Row 19
LTGFTLMGKGVIDGQ

SEQ ID NO: 103 - protein, 15 aa, Cryptomeria japonica
  MISC_FEATURE (1)..(15): Cryj2 peptide, Figure 2, Row 20
LMGKGVIDGQGKQWW

SEQ ID NO: 104 - protein, 15 aa, Cryptomeria japonica
  MISC_FEATURE (1)..(15): Cryj2 peptide, Figure 2, Row 21
VIDGQGKQWWAGQCK

SEQ ID NO: 105 - protein, 15 aa, Cryptomeria japonica
  MISC_FEATURE (1)..(15): Cryj2 peptide, Figure 2, Row 22
GKQWWAGQCKWVNGR

SEQ ID NO: 106 - protein, 15 aa, Cryptomeria japonica
  MISC_FEATURE (1)..(15): Cryj2 peptide, Figure 2, Row 23
AGQCKWVNGREICND

SEQ ID NO: 107 - protein, 15 aa, Cryptomeria japonica
  MISC_FEATURE (1)..(15): Cryj2 peptide, Figure 2, Row 24
WVNGREICNDRDRPT

SEQ ID NO: 108 - protein, 15 aa, Cryptomeria japonica
  MISC_FEATURE (1)..(15): Cryj2 peptide, Figure 2, Row 25
EICNDRDRPTAIKFD

SEQ ID NO: 109 - protein, 15 aa, Cryptomeria japonica
  MISC_FEATURE (1)..(15): Cryj2 peptide, Figure 2, Row 26
RDRPTAIKFDFSTGL

SEQ ID NO: 110 - protein, 15 aa, Cryptomeria japonica
  MISC_FEATURE (1)..(15): Cryj2 peptide, Figure 2, Row 27
AIKFDFSTGLIIQGL

SEQ ID NO: 111 - protein, 15 aa, Cryptomeria japonica
  MISC_FEATURE (1)..(15): Cryj2 peptide, Figure 2, Row 28
FSTGLIIQGLKLMNS

SEQ ID NO: 112 - protein, 15 aa, Cryptomeria japonica
  MISC_FEATURE (1)..(15): Cryj2 peptide, Figure 2, Row 29
IIQGLKLMNSPEFHL

SEQ ID NO: 113 - protein, 15 aa, Cryptomeria japonica
  MISC_FEATURE (1)..(15): Cryj2 peptide, Figure 2, Row 30
KLMNSPEFHLVFGNC

SEQ ID NO: 114 - protein, 15 aa, Cryptomeria japonica
  MISC_FEATURE (1)..(15): Cryj2 peptide, Figure 2, Row 31
PEFHLVFGNCEGVKI

SEQ ID NO: 115 - protein, 15 aa, Cryptomeria japonica
  MISC_FEATURE (1)..(15): Cryj2 peptide, Figure 2, Row 32
VFGNCEGVKIIGISI

SEQ ID NO: 116 - protein, 15 aa, Cryptomeria japonica
  MISC_FEATURE (1)..(15): Cryj2 peptide, Figure 2, Row 33
EGVKIIGISITAPRD

SEQ ID NO: 117 - protein, 15 aa, Cryptomeria japonica
  MISC_FEATURE (1)..(15): Cryj2 peptide, Figure 2, Row 34
IGISITAPRDSPNTD

SEQ ID NO: 118 - protein, 15 aa, Cryptomeria japonica
  MISC_FEATURE (1)..(15): Cryj2 peptide, Figure 2, Row 35
TAPRDSPNTDGIDIF

SEQ ID NO: 119 - protein, 15 aa, Cryptomeria japonica
  MISC_FEATURE (1)..(15): Cryj2 peptide, Figure 2, Row 36
SPNTDGIDIFASKNF

SEQ ID NO: 120 - protein, 15 aa, Cryptomeria japonica
  MISC_FEATURE (1)..(15): Cryj2 peptide, Figure 2, Row 37
GIDIFASKNFHLQKN

SEQ ID NO: 121 - protein, 15 aa, Cryptomeria japonica
  MISC_FEATURE (1)..(15): Cryj2 peptide, Figure 2, Row 38
ASKNFHLQKNTIGTG

SEQ ID NO: 122 - protein, 15 aa, Cryptomeria japonica
  MISC_FEATURE (1)..(15): Cryj2 peptide, Figure 2, Row 39
HLQKNTIGTGDDCVA

SEQ ID NO: 123 - protein, 15 aa, Cryptomeria japonica
  MISC_FEATURE (1)..(15): Cryj2 peptide, Figure 2, Row 40
TIGTGDDCVAIGTGS

SEQ ID NO: 124 - protein, 15 aa, Cryptomeria japonica
  MISC_FEATURE (1)..(15): Cryj2 peptide, Figure 2, Row 41
DDCVAIGTGSSNIVI

SEQ ID NO: 125 - protein, 15 aa, Cryptomeria japonica
  MISC_FEATURE (1)..(15): Cryj2 peptide, Figure 2, Row 42
IGTGSSNIVIEDLIC

SEQ ID NO: 126 - protein, 15 aa, Cryptomeria japonica
  MISC_FEATURE (1)..(15): Cryj2 peptide, Figure 2, Row 43
SNIVIEDLICGPGHG

SEQ ID NO: 127 - protein, 15 aa, Cryptomeria japonica
  MISC_FEATURE (1)..(15): Cryj2 peptide, Figure 2, Row 44
EDLICGPGHGISIGS

SEQ ID NO: 128 - protein, 15 aa, Cryptomeria japonica
  MISC_FEATURE (1)..(15): Cryj2 peptide, Figure 2, Row 45
GPGHGISIGSLGREN

SEQ ID NO: 129 - protein, 15 aa, Cryptomeria japonica
  MISC_FEATURE (1)..(15): Cryj2 peptide, Figure 2, Row 46
ISIGSLGRENSRAEV

SEQ ID NO: 130 - protein, 15 aa, Cryptomeria japonica
  MISC_FEATURE (1)..(15): Cryj2 peptide, Figure 2, Row 47
LGRENSRAEVSYVHV

SEQ ID NO: 131 - protein, 15 aa, Cryptomeria japonica
  MISC_FEATURE (1)..(15): Cryj2 peptide, Figure 2, Row 48
SRAEVSYVHVNGAKF

SEQ ID NO: 132 - protein, 15 aa, Cryptomeria japonica
  MISC_FEATURE (1)..(15): Cryj2 peptide, Figure 2, Row 49
SYVHVNGAKFIDTQN

SEQ ID NO: 133 - protein, 15 aa, Cryptomeria japonica
  MISC_FEATURE (1)..(15): Cryj2 peptide, Figure 2, Row 50
NGAKFIDTQNGLRIK

SEQ ID NO: 134 - protein, 15 aa, Cryptomeria japonica
  MISC_FEATURE (1)..(15): Cryj2 peptide, Figure 2, Row 51
IDTQNGLRIKTWQGG

SEQ ID NO: 135 - protein, 15 aa, Cryptomeria japonica
  MISC_FEATURE (1)..(15): Cryj2 peptide, Figure 2, Row 52
GLRIKTWQGGSGMAS

SEQ ID NO: 136 - protein, 15 aa, Cryptomeria japonica
  MISC_FEATURE (1)..(15): Cryj2 peptide, Figure 2, Row 53
TWQGGSGMASHIIYE

SEQ ID NO: 137 - protein, 15 aa, Cryptomeria japonica
  MISC_FEATURE (1)..(15): Cryj2 peptide, Figure 2, Row 54
SGMASHIIYENVEMI

SEQ ID NO: 138 - protein, 15 aa, Cryptomeria japonica
  MISC_FEATURE (1)..(15): Cryj2 peptide, Figure 2, Row 55
HIIYENVEMINSENP

SEQ ID NO: 139 - protein, 15 aa, Cryptomeria japonica
  MISC_FEATURE (1)..(15): Cryj2 peptide, Figure 2, Row 56
NVEMINSENPILINQ

SEQ ID NO: 140 - protein, 15 aa, Cryptomeria japonica
  MISC_FEATURE (1)..(15): Cryj2 peptide, Figure 2, Row 57
NSENPILINQFYCTS

SEQ ID NO: 141 - protein, 15 aa, Cryptomeria japonica
  MISC_FEATURE (1)..(15): Cryj2 peptide, Figure 2, Row 58
ILINQFYCTSASACQ

SEQ ID NO: 142 - protein, 15 aa, Cryptomeria japonica
  MISC_FEATURE (1)..(15): Cryj2 peptide, Figure 2, Row 59
FYCTSASACQNQRSA

SEQ ID NO: 143 - protein, 15 aa, Cryptomeria japonica
  MISC_FEATURE (1)..(15): Cryj2 peptide, Figure 2, Row 60
ASACQNQRSAVQIQD

SEQ ID NO: 144 - protein, 15 aa, Cryptomeria japonica
  MISC_FEATURE (1)..(15): Cryj2 peptide, Figure 2, Row 61
NQRSAVQIQDVTYKN

SEQ ID NO: 145 - protein, 15 aa, Cryptomeria japonica
  MISC_FEATURE (1)..(15): Cryj2 peptide, Figure 2, Row 62
VQIQDVTYKNIRGTS

SEQ ID NO: 146 - protein, 15 aa, Cryptomeria japonica
  MISC_FEATURE (1)..(15): Cryj2 peptide, Figure 2, Row 63
VTYKNIRGTSATAAA

SEQ ID NO: 147 - protein, 15 aa, Cryptomeria japonica
  MISC_FEATURE (1)..(15): Cryj2 peptide, Figure 2, Row 64
IRGTSATAAAIQLKC

SEQ ID NO: 148 - protein, 15 aa, Cryptomeria japonica
  MISC_FEATURE (1)..(15): Cryj2 peptide, Figure 2, Row 65
ATAAAIQLKCSDSMP

SEQ ID NO: 149 - protein, 15 aa, Cryptomeria japonica
  MISC_FEATURE (1)..(15): Cryj2 peptide, Figure 2, Row 66
IQLKCSDSMPCKDIK

SEQ ID NO: 150 - protein, 15 aa, Cryptomeria japonica
  MISC_FEATURE (1)..(15): Cryj2 peptide, Figure 2, Row 67
SDSMPCKDIKLSDIS

SEQ ID NO: 151 - protein, 15 aa, Cryptomeria japonica
  MISC_FEATURE (1)..(15): Cryj2 peptide, Figure 2, Row 68
CKDIKLSDISLKLTS

SEQ ID NO: 152 - protein, 15 aa, Cryptomeria japonica
  MISC_FEATURE (1)..(15): Cryj2 peptide, Figure 2, Row 69
LSDISLKLTSGKIAS

SEQ ID NO: 153 - protein, 15 aa, Cryptomeria japonica
  MISC_FEATURE (1)..(15): Cryj2 peptide, Figure 2, Row 70
LKLTSGKIASCLNDN

SEQ ID NO: 154 - protein, 15 aa, Cryptomeria japonica
  MISC_FEATURE (1)..(15): Cryj2 peptide, Figure 2, Row 71
GKIASCLNDNANGYF

SEQ ID NO: 155 - protein, 15 aa, Cryptomeria japonica
  MISC_FEATURE (1)..(15): Cryj2 peptide, Figure 2, Row 72
CLNDNANGYFSGHVI

SEQ ID NO: 156 - protein, 15 aa, Cryptomeria japonica
  MISC_FEATURE (1)..(15): Cryj2 peptide, Figure 2, Row 73
ANGYFSGHVIPACKN

SEQ ID NO: 157 - protein, 15 aa, Cryptomeria japonica
  MISC_FEATURE (1)..(15): Cryj2 peptide, Figure 2, Row 74
SGHVIPACKNLSPSA

SEQ ID NO: 158 - protein, 15 aa, Cryptomeria japonica
  MISC_FEATURE (1)..(15): Figure 7, Row a
KSMKVTVAFNQFGPN

SEQ ID NO: 159 - protein, 15 aa, Cryptomeria japonica
  MISC_FEATURE (1)..(15): Figure 7, Row b
PCVFIKRVSNVIIHG

SEQ ID NO: 160 - protein, 15 aa, Cryptomeria japonica
  MISC_FEATURE (1)..(15): Figure 7, Row c
VDGIIAAYQNPASWK

SEQ ID NO: 161 - protein, 20 aa, Cryptomeria japonica
  MISC_FEATURE (1)..(20): Figure 7, Row d
GIDIFASKNFHLQKNTIGTG

SEQ ID NO: 162 - protein, 20 aa, Cryptomeria japonica
  MISC_FEATURE (1)..(20): Figure 7, Row e
LKLTSGKIASCLNDNANGYF

SEQ ID NO: 163 - protein, 15 aa, Cryptomeria japonica
  MISC_FEATURE: Figure 15, p22-1.
PCVFIKRVSNVIIHG

SEQ ID NO: 164 - protein, 14 aa, Cryptomeria japonica
  MISC_FEATURE: Figure 15, p22-2.
CVFIKRVSNVIIHG

SEQ ID NO: 165 - protein, 13 aa, Cryptomeria japonica
  MISC_FEATURE: Figure 15, p22-3.
VFIKRVSNVIIHG

SEQ ID NO: 166 - protein, 12 aa, Cryptomeria japonica
  MISC_FEATURE: Figure 15, p22-4.
FIKRVSNVIIHG

SEQ ID NO: 167 - protein, 11 aa, Cryptomeria japonica
  MISC_FEATURE: Figure 15, p22-5.
IKRVSNVIIHG

SEQ ID NO: 168 - protein, 10 aa, Cryptomeria japonica
  MISC_FEATURE: Figure 15, p22-6.
KRVSNVIIHG

SEQ ID NO: 169 - protein, 14 aa, Cryptomeria japonica
  MISC_FEATURE: Figure 15, p22-7.
PCVFIKRVSNVIIH

SEQ ID NO: 170 - protein, 13 aa, Cryptomeria japonica
  MISC_FEATURE: Figure 15, p22-8.
PCVFIKRVSNVII

SEQ ID NO: 171 - protein, 12 aa, Cryptomeria japonica
  MISC_FEATURE: Figure 15, p22-9.
PCVFIKRVSNVI

SEQ ID NO: 172 - protein, 11 aa, Cryptomeria japonica
  MISC_FEATURE: Figure 15, p22-10.
PCVFIKRVSNV

SEQ ID NO: 173 - protein, 10 aa, Cryptomeria japonica
  MISC_FEATURE: Figure 15, p22-11.
PCVFIKRVSN

SEQ ID NO: 174 - protein, 13 aa, Cryptomeria japonica
  MISC_FEATURE: Figures 17 and 18.
VFIKRVSNVIIHG

(19) United States
(12) Patent Application Publication
(10) Pub. No.: US 2003/0185847 A1
(43) Pub. Date: Oct. 2, 2003

(54) Peptide-based immunotherapeutic agent for treating allergic diseases

(76) Inventors: Toshio Sone, Kanagawa (JP); Akinori Kume, Kanagawa (JP); Kazuo Dairiki, Kanagawa (JP); Akiko Iwama, Kanagawa (JP); Kohsuke Kino, Kanagawa (JP)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/354,240

(22) Filed: Jan. 29, 2003

Related U.S. Application Data
(62) Division of application No. 09/142,524, filed on Sep. 9, 1998.
Application No. 09/142,524 is a 371 of international application No. PCT/JP97/00740, filed on Mar. 10, 1997.

(51) Int. Cl. A61K 39/00; C12N 9/24; C07K 14/415

(52) U.S. Cl. 424/185.1; 435/200; 530/370

(57) ABSTRACT

The present invention provides a monomolecular multi-epitope peptide prepared by bonding T-cell epitope regions derived from different allergen molecules with each other. A peptide-based immunotherapeutic agent containing an effective amount of the multi-epitope peptide can prevent and treat a wide range of allergic diseases.

### TECHNICAL FIELD

The present invention relates to a multi-epitope peptide, which is useful for peptide-based immunotherapy of allergic diseases.

### BACKGROUND ART

Allergic diseases are defined to be functional disturbances caused by type I hypersensitivity (type I immune response mediated by IgE antibodies) or a kind of disease induced by the disturbance. The symptoms include pollinosis, bronchial asthma, allergic rhinitis, atopic dermatitis, and anaphylactic shock. Pollinosis is a representative allergic disease. In Japan, approximately 10% of the population suffers from cedar pollinosis, and the number of the patients is still increasing. In America, 5 to 15% of the population suffers from short ragweed pollinosis. Pollinosis is a serious problem both socially and economically because there are many patients and they suffer from unbearable conditions such as itchiness of eyes, runny noses, sneezing, and nasal congestion. Moreover, once the patient acquires pollinosis, the disease manifests itself every year. An effective therapy for pollinosis has thus earnestly been sought.

To comprehend and treat allergic diseases, it is important to understand how a type I allergic response is developed. Current studies focus on clarifying the initial reaction in the allergen-specific immune response, especially the mechanism of regulating a T cell-mediated allergic reaction. Initiation of an immune response to a foreign antigen including an allergen depends on antigen-presenting cells in the immune system. The antigen-presenting cells (i.e., B cells, macrophages, and dendritic cells) take up incoming foreign antigens, break them down to antigen peptides (T cell epitope peptides), put the fragments in a pocket consisting of α and β chains of major histocompatibility complex (MHC) class II molecules (HLA class II in human), display the fragments on the cell surface, and thereby present the foreign antigens to antigen-specific CD4 positive helper T cells (Th cells). An HLA class II molecule consists of DR, DQ and DP molecules. The α-chain of the DR molecule is encoded by the HLA-DRA gene, and the β-chain is encoded by the HLA-DRB1, -DRB3, -DRB4 or -DRB5 gene. The α-chain of the DQ molecule is encoded by the HLA-DQA1 gene, and the β-chain is encoded by the HLA-DQB1 gene. The α-chain of the DP molecule is encoded by the HLA-DPA1 gene, and the β-chain is encoded by the HLA-DPB1 gene. Each gene except for HLA-DRA contains many alleles. The pocket in which antigenic peptides are placed is highly polymorphic, and the structures differ slightly from each other. Because of this, the kind of antigenic peptides that bind to the pocket and are presented to T cells is restricted to that structure.

Once Th cells receive HLA class II-restricting antigen information via the T cell receptor (TCR), they are activated to secrete various cytokines, by which they proliferate by themselves. At the same time, the Th cells induce differentiation of B cells into plasma cells to induce antibody production. Depending upon the difference in the cytokine-producing pattern, the Th cells activated by antigen stimulation are classified into Th 1 cells capable of producing interferon 2 (IL-2), interferonγ (IFN-γ) and lymphotoxin (TNF-β); Th 2 cells capable of producing IL-4, IL-5, IL-6, IL-10 and IL-13; and Th0 cells capable of producing both cytokines. The production of IgE antibody, which is a cause of allergy, is promoted by IL-4 and IL-13 but suppressed by IFN-γ. That is, Th1 cells suppress IgE production, whereas Th2 cells promote IgE production. In other words, sensitization of allergy is determined by whether Th1 cells or Th2 cells function upon exposure of antigens. It is commonly known that Th2 cells predominantly function in the patients with allergy. Allergen-specific IgE antibodies adhere to peripheral basophil and tissue mast cells. The subsequent exposure of allergen results in cross-linking of the IgE antibody on the basophil or the mast cell via the allergen. This releases inflammatory mediators including histamine, prostaglandins, and leucotriene, thereby causing an immediate allergy response. In response to these inflammatory mediators, lymphocytes, monocytes, basophils, and eosinophils are localized in the inflammatory region of the tissue and result in the release of mediators that cause various reactions including disturbance and a late phase reaction.

One way to treat a particular allergy by antigen-specifically suppressing IgE antibody production is hyposensitization therapy using an allergen protein molecule. Hyposensitization therapy can provide a long-term effect that cannot be achieved by chemotherapy, and hence, is the only treatment close to an effective therapy. However, hyposensitization therapy is not always accepted as a general method for treating allergy, possibly because its mechanism and possible side effects (such as topical swelling or anaphylactic shock) remain unknown.

In place of hyposensitization therapy, a mechanism of hyposensitization using a peptide antigen bearing a T cell epitope has been proposed. The peptide fragment carrying a T cell epitope on the allergen molecule used for this therapy contains no B cell epitope or, if any, is monovalent so that the peptide fails to cross-link an IgE receptor with high affinity on the mast cell. For these reasons, patients administered the peptide fragment should not experience side effects such as anaphylactic shock. It is further known that when T cell epitope is given in vivo, T cells are antigen-specifically inactivated (anergy) (La Salle J. M. et al.:. J. Exp. Med. 176: 177-186, 1992). It is reported that based on such a theoretical background, hyposensitization using a peptide carrying major T cell epitopes of cat dander allergen Fel d1 was carried out in an experimental murine model, and T cell anergy was induced in vitro (Briner, T. J. et al.: Proc. Natl. Acad. Sci. USA, 90: 7608-7612, 1994). Clinical trials on hyposensitization using this peptide are now under way (Norman, P. S. et al.: Am. J. Respir. Crit. Care Med. 154: 1623-1628, 1996; Simons, F. E. et al.: Int. Immunol. 8: 1937-1945, 1996). Hyposensitization therapy using such a peptide carrying the major T cell epitope on the allergen molecule is called “peptide-based immunotherapy” (or “peptide-based hyposensitization therapy”).

As a standard for selecting T cell epitope peptides appropriate for the peptide-based immunotherapy, a positivity index (a mean T cell stimulation index multiplied by appearance frequency) is proposed in WO 94/01560. It is also reported that in peptide design, HLA haplotypic variations in a population of patients should be covered (Wallner, B. P. & Gefter M. L.: Allergy, 49: 302-308, 1994).

### DISCLOSURE OF THE INVENTION

Generally, allergic patients have specific IgE antibodies to each of two or more allergen molecules differing from each other. For a potent allergy therapy, it is important to develop a peptide-based immunotherapeutic agent effective for these patients. However, such an immunotherapeutic agent has not yet been developed. Even the idea of such an agent has never been published in any of the above literatures. Accordingly, an objective of the present invention is to provide a peptide-based immunotherapeutic agent that is efficacious even for allergy patients sensitive to two or more different allergens.

Cedar pollen contains two major allergens, Cry j 1 (Yasueda, H. et al.: J. Allergy Clin. Immunol. 71: 77-86, 1983) and Cry j 2 (Taniai, M. et al.: FEBS Letter 239: 329-332, 1988; Sakaguchi, M. et al.: Allergy 45: 309-312, 1990). More than 90% of the patients with cedar pollinosis possess specific IgE antibodies to Cry j 1 and Cry j 2; the remaining patients (slightly less than 10%) possess a specific IgE antibody to either Cry j 1 or Cry j 2 (Hashimoto, M. et al.: Clin. Exp. Allergy 25: 848-852, 1995). Use of one or more T cell epitopes from only Cry j 1 or Cry j 2 would be expected to be less effective since IgE from the patients is reactive to both Cry j 1 and Cry j 2. Thus, T cell eptopes from both Cry j 1 and Cry j 2 should be chosen to elevate the efficacy of the peptide-based immunotherapy for cedar pollinosis. Therefore, the present inventors prepared a multi-epitope peptide containing T cell epitopes of both Cry j 1 and Cry j 2 in the same molecule. They found that the multi-epitope peptide activated T cells of patients with pollinosis in vitro but did not react with IgE antibodies of the patients. They also found that an immune response was induced in vivo using mice. Based on these new findings, the inventors found that the multi-epitope peptide in this invention is effective as a peptide-based immunotherapeutic agent for patients with cedar pollinosis.

There are many cases of cedar pollinosis that also show clinical symptoms of Japanese cypress pollens. In view of this and based on the above invention, the present inventors prepared a multi-epitope peptide containing the T cell epitopes of Japanese cypress pollen allergen Cha o 1 (Japanese Patent Application No. Hei 8-153527) and the T cell epitopes of cedar pollen allergen Cry j 1 in the same molecule. The multi-epitope peptide activated T cells of both the patients with cedar pollinosis and the patients with Japanese cypress pollinosis, though these T cells do not react with each of the T cell epitopes. The multi-epitope peptides can thus be designed for T cell epitopes derived from not only cedar and Japanese cypress pollen allergens but also other various allergens.

HLA haplotype was investigated in a group of patients (including different races) as a criterion for selecting T cell epitopes to design multi-epitopes that are effective for a broader range of patients. T cell epitope peptides were selected noting that those binding to HLA whose haplotype frequently appears in the population and those presented on different HLA class II molecules, not the same HLA class II molecule, should be selected. The thus-selected multi-epitope peptides were clarified to be effective for a wider range of patients.

The present invention includes the inventions described in each claim.

The present invention will be described below in view of designing of multi-epitope peptides effective for the patients sensitive to cedar pollens or Japanese cypress pollens or the patients sensitive to both pollens, but this invention applies to patients sensitive to other allergens as well. The technical concept of the present invention also applies to plant pollens such as short ragweed (Amb a 1, Amb a 2, Amb a 5, Amb t 5, Amb p 5), Dactylis glomerata (Dac g 2), and Lolium perenne (Lol p 1, Lol p 2, Lol p 3); tree pollens such as Alnus glutinosa (Aln g 1), birch tree or Betula verrucosa (Bet v 1, Bet v 2), mountain cedar (Jun s 1), and juniper tree (Jun v 1); and various other allergens not specifically described herein.

The “multi-epitope peptide” used herein means a peptide molecule prepared by linearly joining peptides containing T cell epitopes derived from different allergen molecules (sometimes referred to as an antigenic peptide or merely as a peptide). In this peptide, a region that is cleaved in vivo is preferably inserted between the T cell epitope-containing peptides to minimize the occurrence of epitope sites that are newly recognized. The multi-epitope peptide is finally broken down to the respective antigenic peptides at the cleavage site. When administered, it can exhibit the effect comparable to that of a mixture of these respective antigenic peptides. The cleavage site may take any structure so long as it undergoes cleavage in vivo. Examples of the cleavage site include an arginine dimer and a lysine dimer that are recognition sequences of cathepsin B, which is an enzyme localized in lysosome.

Designing of the multi-epitope peptide according to the present invention will be described with reference to cedar pollen allergens Cry j 1 and Cry j 2 as examples.

Peripheral lymphocytes collected from the patients with cedar pollinosis are stimulated by Cry j 1 or Cry j 2 to produce the T cell line for individual patient. The T cell line is stimulated by an overlapping peptide consisting of about 15 amino acids, which covers the full-length primary structure of Cry j 1 (WO 94/01560) or Cry j 2 (Komiyama, N. et al.: Biochem. Biophys. Res. Commun. 201: 1201, 1994) to identify the antigenic peptides containing T cell epitopes in the Cry j 1 or Cry j 2 sequence (FIGS. 1 and 2).

Next, typing is performed for HLA class II molecules which bind to these antigenic peptides.

In humans, three different molecules, regions DR, DQ, and DP, exist as gene products of the HLA class II. This suggests that differentiation of T cells would be restricted by antigen-presenting molecules DR, DQ, and DP. The T cell clones established for each patient are used to determine by which locus-derived antigen-presenting molecules the antigenic peptide of Cry j 1 or Cry j 2 is presented. They also determine whether the T cells that have received antigenic peptide information via DR, DQ or DP molecules tend to be differentiated into Th1 cells or Th2 cells. Such a typing is performed using the T cell clone established for individual patients (FIGS. 3 and 4).

FIGS. 3 and 4 clearly show that differentiation into Th1, Th2 or Th0 of the T cells stimulated by the antigenic peptide is not restricted by a specific epitope or a specific combination of HLA molecules. In selecting a peptide for designing the multi-epitope peptide of the present invention, any peptide can be a candidate for the antigenic peptide since any T cell epitope-containing peptide can stimulate T cells.

The criteria for selecting peptides to design the multi-epitope peptide of the present invention are as follows:

(1) Peptides are selected in the order of a positivity index (WO 94/01560) (peptides having a positivity index of 100 or more should be selected).

(2) Peptides presented on HLA class II molecules that frequently appear as antigen-presenting molecules are selected.

(3) Where there is no significant difference in the positivity index, peptides presented by restriction molecules of different types are selected to enhance the effectiveness. Specifically, in selecting a T cell epitope of an allergen that causes a certain allergic disease, the HLA haplotype in a group of patients with the allergy is first examined, and a T cell epitope restricted by an HLA haplotype whose gene frequency is high in the population to which the patient group belongs is selected. This is then the best selection that should achieve the best effect in that patient group. However, the thus-selected T cell epitope may not be effective at all in other patient groups.

Taking HLA haplotype DPB1*0501 as an example, it is assumed that this HLA haplotype is quite frequently observed in Japanese patients with a certain allergic disease, and the HLA haplotype-restricting T cell epitope is selected. The thus-selected peptide would hardly be effective for Northern American patients with the same allergy because the gene frequency of the HLA haplotype is as much as 39.0% in Japanese patients, whereas it is as little as 1.3% in white Americans and 0.8% in African Americans in Northern America. For Northern American patients, the HLA-DP restricting T cell epitope DPB1*0401 (in Northern America, 30.2% for white American patients and 11.1% for African American patients; 4.8% for Japanese patients) should be selected. It is also important to select a peptide presented on the antigen-presenting molecules differing in the locus level like DR, DQ, or DP; even though the loci are the same, it is important to select a peptide presented on the antigen-presenting molecules having different haplotypes.

In this case, the preferable epitope site contains no cysteine residue. When the epitope site contains a cysteine residue, the residue might bind non-specifically to HLA class II molecules. When immunized with an antigenic peptide containing a cysteine residue, the site that is originally not an antigen might be recognized as a new epitope. When such a peptide is recognized as an epitope, the cysteine-containing epitope is recognized by the peptide repeatedly through its second and third administrations, which may possibly cause side effects.

Specific embodiments of designing the multi-epitope will be described below. According to the positivity index of Cry j 1 and Cry j 2 shown in FIGS. 1 and 2, the T cell epitope of Cry j 1, Peptide No. 43 with amino acid residues at positions 211-225 (hereinafter abbreviated as p211-225) (restriction molecules DPA1*0101 to DPB1*0501) shows the highest positivity index and Peptide No. 22, p106-120 (restriction molecule DRB5*0101) shows the second highest positivity index. These two peptides are selected as the antigenic peptides to be used in the multi-epitope peptide. Turning to Cry j 2, Peptide No. 14, p66-80 (restriction molecule DRB5*0101) and Peptide No. 38, p186-190 (DRB4*0101) show the highest positivity indexes. Likewise, these two peptides can be selected as the antigenic peptides. Peptide No. 37, p181-195, located before Peptide No. 38 in Cry j 2 has a high positivity index of 280, but its restriction molecules are DPA1*0101 to DPB1*0201, which differ from the restriction molecule of Peptide No. 38. Since Peptide No. 37, p181-195 overlaps with Peptide No. 38, p186-200 by 10 residues, 5 residues from No. 37 are added ahead to No. 38. The thus-designed peptide can be selected as an HLA-DP restricting peptide. Peptides as selected above do not restrict DQ. Restriction molecules for Peptide No. 4, p16-30 of Cry j 1, are DQA1*0102 to DQB1*0602, but a cysteine residue is contained at the center of the epitope. Thus, Peptide No. 4 cannot be selected. In Cry j 2, p341-360, corresponding to Peptide Nos. 69-70, is a peptide presented on DQA1*0102 to DQB1*0602. Peptide No. 70 also contains cysteine, whereas T cells can be activated by only the cysteine-free Peptide No. 69. Thus, 12 residues, p344-355 (ISLKLTSGKIAS), can be selected. Peptide No. 22, p106-120 of Cry j 1 contains cysteine at position 107. At least nine residues of p109-117 (FIKRVSNVI) are required for determining the T cell epitope core sequence using a T cell clone. Thus, if Pro-Cys residues at p106-107 are removed, the remaining peptide can be used.

The antigen taken up into the antigen-presenting cells is degraded in lysosome. How the foreign proteins taken up into the antigen-presenting molecules are processed and how they are bound to HLA class II molecules are still unknown. However, it is reported that cathepsin B participates in the digestion of antigens in this complicated mechanism (Katsunuma, N.: Nihon Men-Eki Gakkai (Japanese Society of Immunology) 25: 75, 1995).

With respect to several HLA class II types, an HLA-binding amino acid motif of the antigenic peptide has been determined. Binding to HLA class II molecules has specificity, but numerous antigenic peptides can bind to specific HLA class II molecules if the peptides meet a certain criterion (Rammensee, H. G. et al. Immunogenetics. 41: 178-228, 1995). For this reason, a newly recognized epitope site might possibly be created in the antigenic peptide-binding site. To avoid this, the multi-epitope peptide should be designed so as to be cleaved into each of the antigenic peptides in the antigen-presenting cells. The peptide sequence recognized by cathepsin B is the Arg-Arg-hydrophobic sequence or the Lys-Lys-hydrophobic sequence. Therefore, Arg-Arg or Lys-Lys is added to the latter half of the peptide containing the epitope and, in the following epitope sequence, a hydrophobic amino acid sequence is placed after Arg-Arg or Lys-Lys.

Since Arg-Arg is inserted between the antigenic peptides, the order of the antigenic peptides in this specific embodiment is considered insignificant. When Arg is linked to the latter half of Peptide No. 14 of Cry j 2 (FIG. 2), however, Tyr at position 73 becomes a first anchor. The Arg residue added then becomes amino acid residue at position 9 in the peptide motif of DRB5*0101 and serves as a second anchor. Thus, the Arg residue may be recognized as a new epitope. Therefore, this sequence should be located at the end of the multi-epitope peptide.

The thus-obtained multi-epitope peptide is shown as SEQ NO: 1. The restriction molecules for this multi-epitope are DRB4*0101, DRB5*0101, DPA1*0101-DPB1*0201, DPA1*0101-DPB1*0501, and DQA1*0102-DQB1*0602. In The 11th International Histocompatibility Workshop, the frequency of these genes was calculated in the Japanese population (Tsuji, K. et al.: HLA 1991, vol. 1, 1992, Oxford University Press) and found to be 0.291 for DRB4*0101, 0.056 for DRB5*0101 (0.070 for DRB5*0102), 0.208 for DPB1*0201, 0.399 for DPB1*0501, and 0.053 for DQB1*0602 (0.204 for DQB1*0601). Based on these data, the antigen frequency is calculated to be 0.50 for DRB4*0101, 0.11 for DRB5*0101 (0.14 for DRB5*0102), 0.37 for DPB1*0201, 0.64 for DPB1*0501, (0.79 according to Hori et al.), and 0.10 for DQB1*0602 (0.37 for DQB1*0601). Since DRB5*0101 and DQB1*0602 are regarded as identical due to the presence of linkage disequilibrium, the data of DRB5*0101 is used for DQB1*0602. The probability that the Japanese population carries both DPB1*0201 and DPB1*0501 or either one is calculated to be 0.85. Similarly, the probability that the Japanese population carries both of DRB4*0101 and DRB5*0101 or either one is calculated to be 0.56. From these values, about 90% of patients are estimated to recognize more than one T cell epitope contained in the multi-epitope peptide of SEQ NO: 1. However, it is unclear whether the patients with these HLA types possess a T cell repertory capable of recognizing these epitope peptides presented on these restriction molecules. Furthermore, the number of epitopes that cause proliferation of T cells is unknown (two or more epitopes would be necessary). Thus, the efficiency of the multi-epitope peptide might decrease. In practice, it is properly assumed to be approximately 77% based on the result of testing proliferation response of peripheral lymphocytes from 17 patients.

To increase the range of patients to be effectively treated, the multi-epitope peptide can also be designed to carry more T cell epitopes than described above. Examples of such multi-epitope peptides include one prepared by joining p213-225 and p108-120 of Cry j 1, p182-200 and p79-98 of Cry j 2, p80-95 of Cry j 1, and p66-80 of Cry j 1, in this order (SEQ NO: 2), and one prepared by joining p213-225 and p108-120 of Cry j 1, p182-200 and p79-98 of Cry j 2, p67-95 of Cry j 1, and p238-251 and p66-80 of Cry j 2, in this order (SEQ NO: 3). These multi-epitope peptides are effective as peptide-based immunotherapeutic agents since the peptides stimulated all the peripheral lymphocyte samples from the 21 tested patients with cedar pollinosis but did not react with the IgE antibody of the patients. Developing this concept, the effectiveness can be improved by preparing a T cell epitope containing allergens of different species, e.g., both Japanese cypress pollen allergen and cedar pollen allergen, by the method described in Example 13.

The present invention also includes modification of the antigenic peptide region used in the multi-epitope peptide to regulate the activity of T cells. The “modification” used herein means substitution, deletion, and insertion of at least one amino acid residue. Changes of properties of T cells imparted by amino acid substitution in the antigenic peptide can be examined by known methods. For example, 1) a certain amino acid of the multi-epitope peptide of the present invention is substituted with an analogous amino acid, e.g., by substituting Asp with Glu, Asn with Gln, Lys with Arg, Phe with Tyr, Ile with Leu, Gly with Ala, and Thr with Ser, to produce analog peptides, which are compared with the original peptide in T cell proliferating ability or lymphokine-producing ability. Alternatively, 2) a certain amino acid of the multi-epitope peptide is substituted with a non-analogous amino acid, for example, by substituting a polar amino acid or a hydrophilic amino acid with a hydrophobic amino acid Ala, and a hydrophobic amino acid with a hydrophilic amino acid Ser, and the property of the modified peptide is compared to that of the original peptide. The present invention also includes the thus-prepared multi-epitope analog peptides that are immunologically equivalent to the multi-epitope peptide of the present invention in terms of the positivity index and the T cell activation ability.

Most T cells that react with the antigenic peptide derived from Cry j 1 or Cry j 2 possess the properties of Th2 and Th0 in combination (FIGS. 3 and 4). BCG vaccine can potentiate the cellular immune activity to prevent infection with tubercle bacillus. To potentiate cellular immunity, T cells of Th1 type should be induced. It is reported that studies on the property of a human T cell clone with BCG inoculation revealed an increased level of Th1 type T cells (Matsushita, Sho, The 45th Japanese Association of Allergy, 836, 1995) According to Matsushita, there is a Th1 clone that is restricted by HLA-DR14 (DRB1*1405) and that recognizes 84-100 amino acid sequence (EEYLILSARDVLAVVSK) of BCGa protein. If the HLA haplotype DPA1-DPB1*0501-restricting T cell epitope that is possessed by more than 60% of Japanese population is selected (for example, Peptide No. 43 (p211-225)/KSMKVTVAFNQFGPN of Cry j 1 shown in FIG. 1), this peptide is bound to the 84-100 T cell epitope of tubercle bacillus BCGa protein restricted by DRB1*1405. It is highly likely that the thus-prepared multi-epitope peptide EEYLILSARDVLAVVSKRRMKVTVAFNQFGPN would be quite efficacious for patients with cedar pollinosis carrying haplotype DRB1*1405. The use of such a multi-epitope peptide would lead to production of Th1 lymphokines, especially IL-12, by a peptide derived from BCGa antigen. It is known in several cases in humans and mice that IL-12 has an activity contradictory to that of IL-4 and acts on T cells to induce differentiation of Th cells to Th1 (Manetti, R., et al.: J. Exp. Med., 177, 1199-1204, 1993; Wu, C., et al.: J. Immunol., 151, 1938-1949, 1993; Hsieh, C., et al.: Science, 260, 547-549, 1993). In particular, the experimental results by Manetti et al. indicate that a T cell clone specific to Der p1 antigen, a mite allergen, basically induces Th2 but induces Th1 or Th0 in the presence of IL-12. Thus, using the multi-epitope peptide prepared by joining a T cell epitope having Th1 induction activity to an allergen-reactive T cell epitope, T cells that are inherently induced to Th2 would be induced to Th1 or Th0.

When the peptide of the present invention containing at least one T cell epitope of Cry j 1 and/or Cry j 2 is subcutaneously administered to a mouse, which is then exposed to cedar pollen allergen, T cell anergy occurs (FIGS. 13 and 14), and IL-2 production is significantly reduced as compared to the control group. It is reported that hyposensitization therapy reduces IL-2 in humans (J. Allergy Clin. Immunol. 76: 188, 1985). Furthermore, the multi-epitope peptide of the present invention can activate each of the peptide-constituting T cell clones to the T cell epitope peptides (FIG. 10) but does not react with IgE antibodies of the patients (FIG. 8). These results show that the multi-epitope peptide of the present invention induces immune tolerance against allergens and is effective as a peptide-based immunotherapeutic agent for allergic diseases. The multi-epitope peptide of the present invention may be administered together with pharmaceutically acceptable carriers or diluents. The effective dose of the multi-epitope peptide may vary depending upon sensitivity to cedar pollen allergen, age, sex, and the body weight of the patients and other factors such as ability of a peptide to induce immune response in the patients.

The multi-epitope peptide-may be administered in a simple manner using an administration route including injection (subcutaneous or intravenous), rhinenchysis, instillation, oral administration, inhalation, percutaneous administration, etc.

The one-letter notation for amino acids used in the specification and the sequence listing follows the definition prescribed by IUPAC, Commission on Biochemical Nomenclature (cf., Biochemical Dictionary, 2nd ed., 1468, Table 1.1).

### BRIEF DESCRIPTION OF DRAWINGS

FIG. 1 shows a mean stimulation index, frequency of appearance, and a positivity index (mean stimulation index multiplied by frequency of appearance) of the cell line derived from the patients with cedar pollinosis, against Cry j 1 overlapping peptides.

FIG. 2 shows a mean stimulation index, frequency of appearance, and a positivity index (mean stimulation index multiplied by frequency of appearance) of the cell line derived from the patients with cedar pollinosis, against Cry j 2 overlapping peptides.

FIG. 3 shows the Th type of the T cell clones that recognize complexes between the Cry j 1 antigenic peptides and HLA class II molecules as well as the Th types of the HLA class II molecules.

FIG. 4 shows the Th type of T cell clones that recognize complexes between the Cry j 2 antigenic peptides and HLA class II molecules as well as the Th types of the HLA class II molecules.

FIG. 5 shows the results of identifying HLA class II molecules capable of binding to an antigenic peptide at the locus level (DR, DQ, and DP).

FIG. 6 shows the results of identifying HLA class II molecules capable of binding to an antigenic peptide at an allelic level of each locus.

FIG. 7 shows the amino acid sequences used in the multi-epitope peptide. In this figure, Peptides a and b correspond to Peptide Nos. 43 and 22 of Cry j 1, respectively; Peptide c corresponds to No. 14 of Cry j 2; and Peptides d and e correspond to Peptide Nos. 37-38 (p181-200) and Nos. 69-71 (p346-365), respectively.

FIG. 8 shows the reactivity of the multi-epitope peptides designated as C.A.#1, C.A.#2, C.A.#3, C.A.#4, C.A.#5 and C.A.#6 with human IgE.

FIG. 9 shows the results of recognizing the T cell epitopes contained in the multi-epitope peptide C.A.#4 by T cell clones.

FIG. 10 shows the ability of lymphocyte proliferation response of the peripheral lymphocytes of the patients with cedar pollinosis and healthy subjects induced by stimulation with the multi-epitope peptide (SEQ NO: 1) in various concentrations.

FIG. 11 shows the ability of proliferation response of the peripheral lymphocytes of two healthy subjects and 17 patients with cedar pollinosis induced by stimulation with the multi-epitope peptide SEQ NO: 1.

FIG. 12 shows the immune tolerance induced by administration of cedar pollen allergen Cry j 1 to CBF1 mice.

FIG. 13 shows the immune tolerance induced by administration of Peptide No. 14 (p66-80) of Cry j 2 to CBF1 mice.

FIG. 14 shows the immune tolerance induced by administration of Peptide No. 48 (p236-250) of Cry j 2 to CBF1 mice.

FIG. 15 shows core amino acid sequencing of Peptide No. 22 (p106-120) of Cry j 1.

FIG. 16 shows the reactivity of T cell lines of the patients with cedar pollinosis and the patients with hinoki pollinosis with the multi-epitope peptide prepared by binding a cedar pollen-specific T cell epitope peptide to a Japanese cypress pollen-specific T cell epitope peptide.

FIG. 17 shows the proliferation response of T cell clone PJ7-9 to an amino acid-substituted analog peptide of Cry j 1 #22 core peptide and the amount of cytokine subsequently produced.

FIG. 18 shows the proliferation response of T cell clone PB10-18 to the above-described analog peptide and the amount of cytokine subsequently produced.

### BEST MODE FOR IMPLEMENTING THE INVENTION

### Example 1

Identifying T Cell Epitope of Cry j 1 and Cry j 2 Using T Cell Line

Peripheral lymphocytes from 18 patients with cedar pollinosis were stimulated by cedar pollen allergen Cry j 1 or Cry j 2 to establish the T cell line of each patient capable of specifically recognizing the respective allergen.

A mixture of 5×10⁴ cells of the autologous B cell line treated with mitomycin C, 2 μM of an overlapping peptide, and 2×10⁴ cells of the T cell line was incubated for 2 days in RPMI-1640 medium supplemented with 0.2 ml of 15% serum on a 96-well culture plate. After 0.5 μCi [³H] thymidine was added to the medium, incubation was continued for a further 18 hours. After the cells were harvested on a glass filter using a cell harvester, the level of [³H] thymidine taken up into the cells was determined with a liquid scintillation counter. If the stimulation index is 2 or more, we consider that the added peptide is recognized as an antigenic peptide. The stimulation index means a value obtained by dividing the level of [³H] thymidine taken up into the cells when the peptide was added by the level of [³H] thymidine taken up into the cells when no peptide was added.

For Cry j 1, the number of T cell epitopes on the Cry j 1 molecule that each patient recognized was on average 9.8 and ranged from 4 to 15. For Cry j 2, the number of T cell epitopes was on average 8.7 and ranged from 2 to 13. Cry j 1 consists of 353 amino acids, and, Cry j 2, 379 amino acids. Therefore, it was estimated that 2.3 to 2.8 T cell epitopes are present per 100 amino acid residues.

The HLA class II type is considered to vary in every patient. It is thus assumed that a T cell epitope to be recognized would vary depending on the HLA class II type. For this reason, the antigenic peptide that the patients recognized was mapped for the individual patient. The results indicate that the epitopes on the Cry j 1 and Cry j 2 molecules differ depending on the patient. On the allergen molecule, there are both regions that can be readily recognized and regions that can hardly be recognized, as a T cell epitope, depending on individuals. Moreover, since the proliferation rate of T cells varies depending on a T cell epitope, the epitope map alone makes it difficult to determine what antigenic peptide should be chosen to design the multi-epitope peptide. Therefore, eighteen patients were further examined with respect to the antigenic peptide which showed a stimulation index of 2 or more. A mean stimulation index of the antigenic peptide was calculated and multiplied by the rate of patients carrying the antigenic peptide (frequency in appearance) to calculate the “positivity index” which shows the predominant order for the respective epitopes (cf. WO 94/01560).

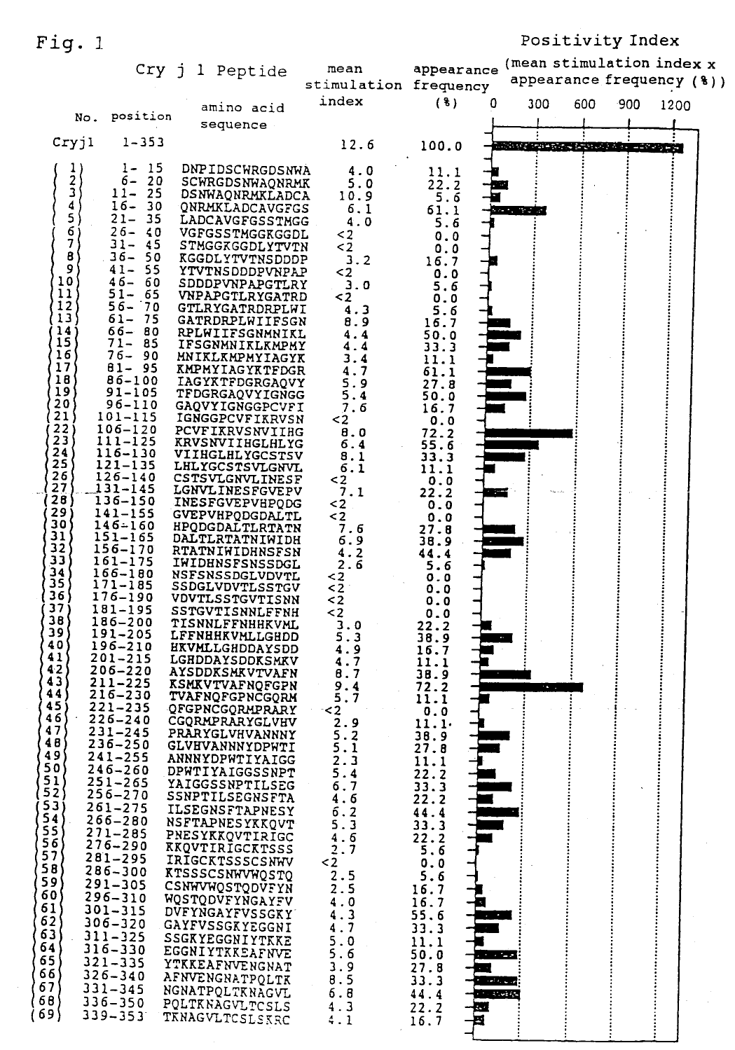

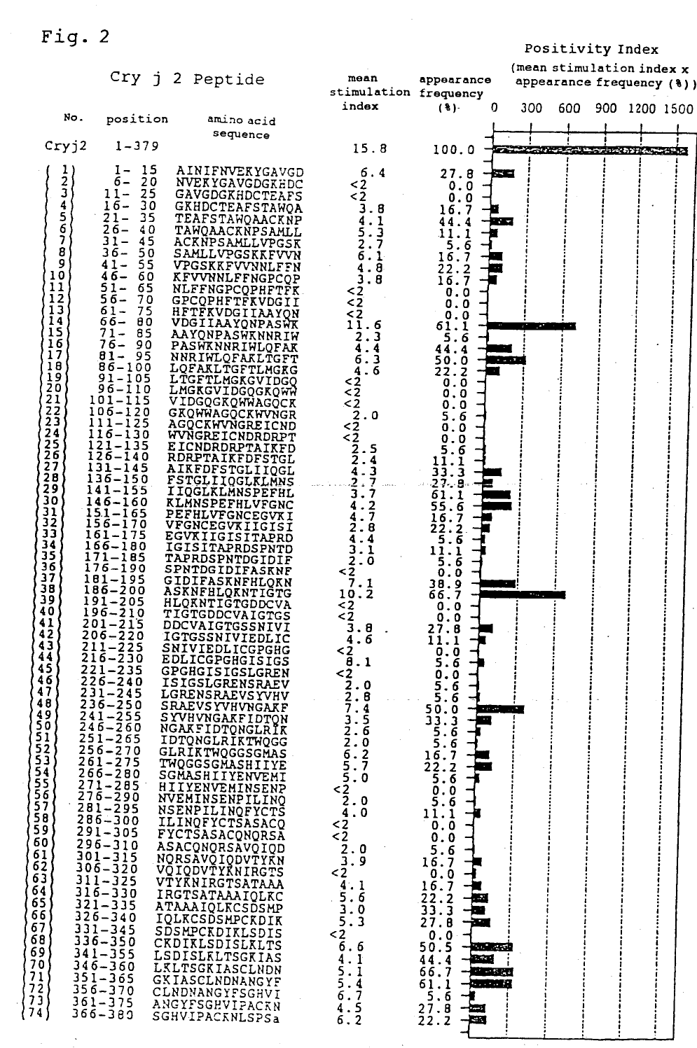

The results are shown in FIGS. 1 and 2. In Cry j 1, Peptide No. 43 (p211-225) shows the highest positivity index, 679, which is followed by the second highest Peptide No. 22 with a positivity index of 578 and Peptide No. 4 with a positivity index of 373. In Cry j 2, Peptide No. 14 shows the highest positivity index (709). Peptide No. 38 with a positivity index of 680 and Peptide No. 48 with a positivity index of 370 then follow. One antigenic peptide having a high positivity index may be selected and used for the peptide-based immunotherapy. However, even for the highest appearance frequency, the effect can be theoretically expected in only 72% of the patients, and the actual efficiency would be lower. To increase the efficiency, it is necessary to use numerous T cell epitopes in combination. In this case, T cell epitopes with a high positivity index are chosen as candidates. However, just using epitopes with a high positivity index alone cannot increase the efficiency if HLA class II molecules presenting these epitopes as antigens are the same. It is thus necessary to identify the type of HLA class II molecules presenting T cell epitope peptides.

### Example 2

Identifying T Cell Epitope Peptide Recognized by T Cell Clone

Two patients, Patient B (PB) and Patient J (PJ), who recognize Peptide Nos. 43 and 22 showing a high positivity index in Cry j 1 and three patients, PB, Patient C (PC), and Patient R (PR), who recognize Peptide Nos. 14, 38, 48, and 69 showing a high positivity index in Cry j 2 were selected from the eighteen patients with cedar pollinosis. Peripheral lymphocytes from these patients with cedar pollinosis were stimulated by Cry j 1 or Cry j 2 to establish T cell clones capable of recognizing Cry j 1 or Cry j 2. The types of HLA class I and class II molecules of the four patients are shown below.

| | |
|---|---|
| PB: | A2/24 - B39/55 - Cw7/w3 - DRB1*1501/0901 - DRB4*0101- |
| | DRB5*0101, DQA1*0102/0301 - DQB1*0602/0303 - |
| | DPA1*0101/0101 - DPB1*0501/0201; |
| PJ: | A24/- - B61/51 - Cw3/- - DRB1*1501/0802 - DRB5*0101, |
| | DQA1*0102/0401 - DQB1*0602/0402 - DPA1*-/- - |
| | DPB1*0501/0402; |
| PC: | A-2/2 - B54/51 - Cw1/-, DRB1*0405/1501 - DRB4*0101 - |
| | DRB5*0101 - DQA1*0301/0102 - DQB1*0401/0602 - |
| | DPA1*0202/0202 - DPB1*0201/0501; |
| PR: | A-11/- - B60/35 - Cw7/w3 - DRB1*0901/1501 - DRB4*0101 - |
| | DRB5*0101 - DQA1*0301/0102 - DQB1*0303/0602 - |
| | DPA1*01/0202 - DPB1*0201/0201. |

Thirty-five T cell clones in total that specifically recognize Cry j 1 were established from the peripheral lymphocytes derived from PB, and 14 similar T cell clones from PJ. Likewise, 31 T cell clones, 10 T cell clones, and 17 T cell clones in total that specifically recognize Cry j 2 were established from the peripheral lymphocytes derived from PB, PC and PR, respectively. Since these T cell clones were all CD3*, CD4⁺, CD8⁻, TCRαβ⁺ and TCRγδ⁻, the restriction molecules were found to be HLA class II molecules. A mixture of 5×10⁴ cells of the autologous B cell line previously treated with mitomycin C, 2 μM of an overlapping peptide, and 2×10⁴ cells of the T cell clone was incubated for 2 days in RPMI-1640 medium supplemented with 0.2 ml of 15% serum on a 96-well micro culture plate. After 0.5 μCi [³H] thymidine was added to the medium, incubation was continued for a further 18 hours. After the cells were harvested on a glass filter using a cell harvester, the level of [³H] thymidine taken up into the cells was determined using a liquid scintillation counter. By this procedure, the T cell epitope recognized by each of the T cell clones was identified.

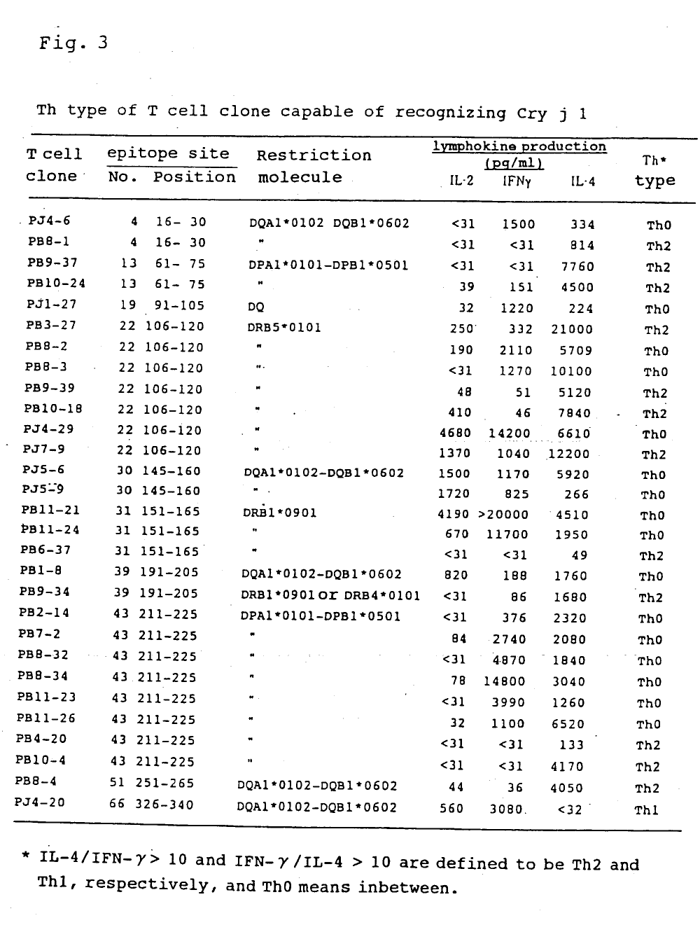

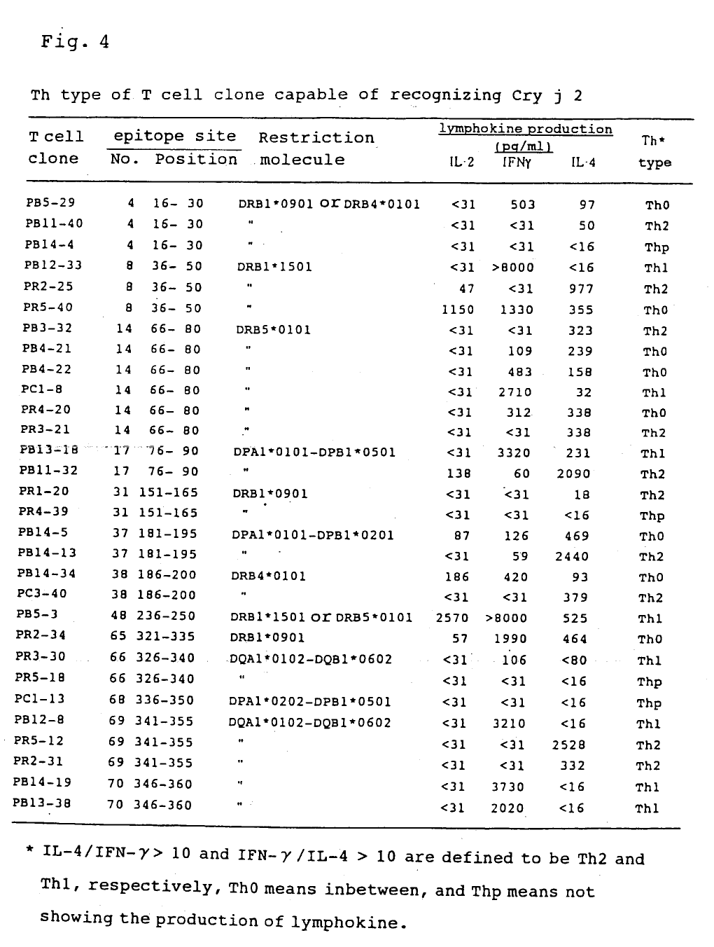

In the T cell clones that recognized Cry j 1, 69% (34/49) showed a proliferation response by stimulation with the peptides and, as a result, the epitopes were identified. Similarly, the antigenic peptide could be identified in 69% (40/58) out of the T cell clones which recognized Cry j 2. The T cell clones capable of specifically recognizing Cry j 1 recognized Peptide Nos. 4, 13, 19, 22, 30, 31, 39, 43, 51, and 66, and the T cell clone capable of specifically recognizing Cry j 2 recognized Peptide Nos. 4, 8, 14, 17, 31, 37, 38, 48, 65, 66, 68, 69, and 70. The results are summarized in FIGS. 3 and 4.

### Example 3

Identifying HLA Class TT Restriction Molecules at the Locus Level

HLA class II restriction molecules were identified at the locus level by adding a monoclonal antibody capable of specifically reacting with DR, DQ or DP of HLA class II molecules to the proliferation response system of the T cell clones established in Example 2, thereby inhibiting the proliferation response of T cells.

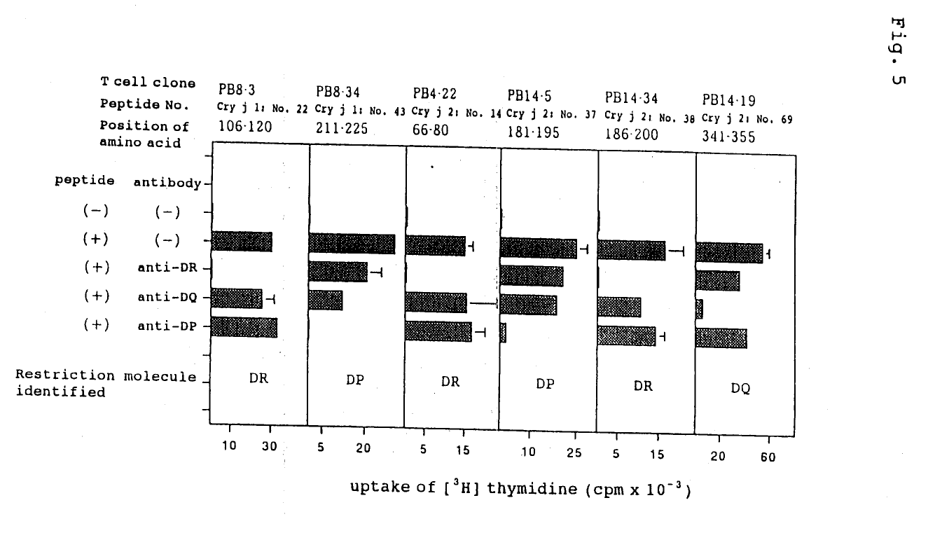

A mixture of 2×10⁴ cells of the autologous B bell line previously treated with mitomycin C; 2 μM of an overlapping peptide; 3 μg/ml of anti-DR, -DQ or -DP monoclonal antibody (manufactured by Becton Dickinson Inc.); and 2×10⁴ cells of the T cell clone was incubated for 2 days in RPMI-1640 medium supplemented with 0.2 ml of 15% serum on a 96-well micro culture plate. After 0.5 μCi [³H] thymidine was added to the medium, incubation was continued for a further 18 hours. After the cells were harvested on a glass filter using a cell harvester, the level of [³H] thymidine taken up into the cells was determined using a liquid scintillation counter. The results shown in FIG. 5 indicate that the restriction molecule of the Cry j 1 p106-120, Cry j 2 p66-80 and Cry j 2 p186-200 peptides was DR; that of the Cry j 2 p341-355 peptide was DQ; and that of the Cry j 1 p211-225and Cry j 2 p181-195was DP. The restriction molecules of other T cell clones were analyzed in the same manner (cf. FIGS. 3 and 4).

### Example 4

Identifying the HLA Class TT Restriction Molecules

HLA class II restriction molecules can be identified using the T cell clones whose restriction molecules were identified at the HLA class II locus level and, as antigen-presenting cells, mouse L-cells transfected with each type for DR and B cell line having the same haplotype for DQ or DP.

A mixture of 5×10⁴ mouse L cells previously treated with mitomycin C or the B cell line coincident in haplotype; 2 μM of an overlapping peptide; 3 μg/ml of anti-DR, -DQ or -DP monoclonal antibody (manufactured by Becton-Dickinson Inc.); and 2×10⁴ cells of the T cell clone was incubated for 2 days in RPMI-1640 medium supplemented with 0.2 ml of 15% serum on a 96-well micro culture plate. After 0.5 μCi [³H] thymidine was added to the medium, incubation was continued for a further 18 hours. After the cells were harvested on a glass filter using a cell harvester, the level of [³H] thymidine taken up into the cells was determined using a liquid scintillation counter.

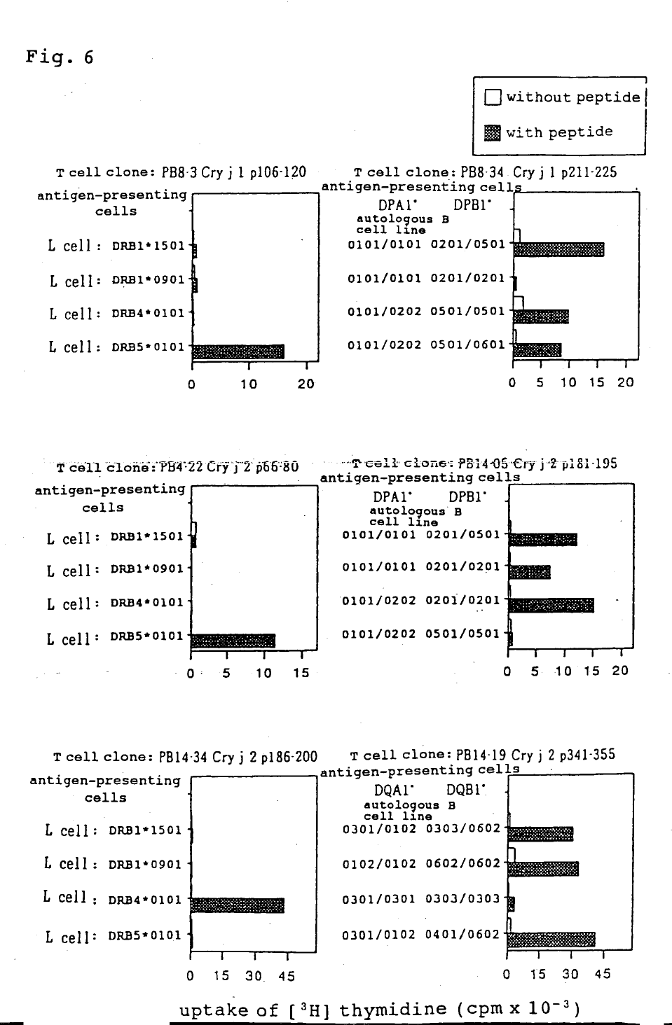

The restriction molecules can be identified by observing the proliferation response of the T cell clones. The Cry j 1 p106-120 peptide-presenting restriction molecule was DRB5*0101, the Cry j 1 p211-225 peptide-presenting restriction molecule was DPA1*0101-DPB1*0501, the Cry j 2 p66-80 peptide-presenting restriction molecule was DRB5*0101, the Cry j 2 p181-195 peptide-presenting restriction molecules was DPA1*0101-PDB1*0201, the Cry j 2 p186-200 peptide-presenting restriction molecules was DRB4*0101, and the Cry j 2 p341-355 peptide-presenting restriction molecules was DQA1*0102-DQB1*0602 (FIG. 6). The results obtained with the other epitope sites are shown in FIGS. 3 and 4.

### Example 5

Identifying the Th Type of T Cell Clone

Th2 cells are considered to participate in the development of allergy. The current level of investigations has not completely clarified if differentiation of T cells into Th1 or Th2 cells is restricted, after antigen stimulation, by a specific epitope peptide or on a HLA class II locus level. When Th2 cells are predominantly induced after stimulation with a peptide, it is highly likely that administration of the peptide will worsen the cedar pollinosis. The T cell clones prepared in Example 2 were stimulated with the epitope peptide recognized by T cells. Th type was determined by measuring the amount of IL-2, IL-4, and IFN-γ produced.

A mixture of 1×10⁵ cells of the autologous B cell line previously treated with mitomycin C, 2 μM of the epitope peptide, and 5×10⁵cells of the T cell clone was incubated for 24 hours in RPMI-1640 medium supplemented with 1 ml of 10% human serum on a 24-well micro culture plate. The cells were precipitated by centrifugation to obtain the culture supernatant. IL-2, IL-4, and IFN-γ in the supernatant were determined using the respective ELISA kits commercially available [for IL-2, manufactured by R & D Inc.; for IL-4, manufactured by Medgenics Inc.; and for IFN-γ, manufactured by Otsuka Assay Research Laboratories).

The amounts of IL-2, IL-4, and IFN-γ produced by each T cell clone are shown in FIGS. 3 and 4. The T cell clones which recognize Cry j 1 were twelve Th2, one Th1, and sixteen Th0 cells, showing that there were more Th2 clones than Th1 clones. In contrast, the T cell clones which recognize Cry j 2 were ten Th2, eight Th1, and eight Th0 cells, showing that the number of Th2 clones was roughly equal to the number of Th1 clones. A comparison of T cell epitopes recognized by the respective T cell clones, restriction molecules, and Th type reveals that the Th2, Th1 or Th0 type varies depending upon each T cell clone. Both Th2 cells and Th1 cells are found in a few T cell clones which recognize the same epitope and the same antigen-presenting molecule. These results indicate that after stimulation with Cry j 1 or Cry j 2, differentiation of T cells into Th2, Th1 or Th0 is not controlled by the combination of a specific T cell epitope and a specific restriction molecule. In other words, all of the peptides carrying the T cell epitope sites can be candidates for the multi-epitope peptide of the present invention.

### Example 6

Preparing the Multi-Epitope Peptide

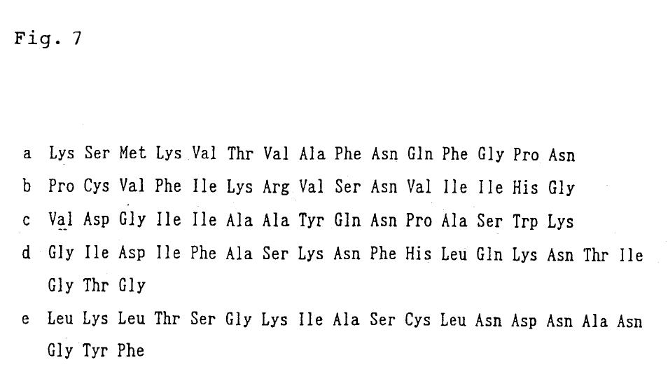

Identifying the IgE antibody epitope sites present on Cry j 1 and Cry j 2 reveals that Cry j 1 lacks an IgE epitope capable of recognizing the primary structure and at least four IgE antibody epitope sites are present on Cry j 2. However, these IgE antibody epitope sites differ from the epitope sites of T cells. Based on this finding, the peptides shown in FIG. 7 were selected from the T cell epitopes of Cry j 1 and Cry j 2.

Peptides a and b shown in FIG. 7 correspond respectively to Peptide Nos. 43 and 22 of Cry j 1 shown in FIG. 1, Peptide c corresponds to No. 14 of Cry j 2 shown in FIG. 2, and Peptides d and respectively consist of a part of the amino acids 37-38 and 69-71 of Cry j 2 shown in FIG. 2.

These six peptides were joined to each other in tandem to prepare the multi-epitope peptide of the present invention. In this case, the two peptides a and b were joined in the order of a and then b the remaining three peptides (Peptides c, d and e) were joined at random. The sequence Arg-Arg was inserted between the peptides. Thus, the following six multi-epitope peptides were produced:

| | |
|---|---|
| C.A. #1. | a-Arg-Arg-b-Arg-Arg-c-Arg-Arg-d-Arg-Arg-e |
| C.A. #2. | a-Arg-Arg-b-Arg-Arg-c-Arg-Arg-e-Arg-Arg-d |
| C.A. #3. | a-Arg-Arg-b-Arg-Arg-d-Arg-Arg-c-Arg-Arg-e |
| C.A. #4. | a-Arg-Arg-b-Arg-Arg-d-Arg-Arg-e-Arg-Arg-c |
| C.A. #5. | a-Arg-Arg-b-Arg-Arg-e-Arg-Arg-c-Arg-Arg-d |
| C.A. #6. | a-Arg-Arg-b-Arg-Arg-e-Arg-Arg-d-Arg-Arg-c |

### Example 7

Reactivity of the Multi-Epitope Peptides with Human TgE Antibody

The six multi-epitope peptides (C.A.#1 through C.A.#6) obtained in Example 6 were dissolved in 0.2 M acetate buffer solution (pH 4.5). The solution was dispensed in quantities of 0.1 ml/well in a black plate (manufactured by Dainihon Pharmaceutical Co., Ltd.) then allowed to stand at 4° C. overnight. After the antigen solution was removed, the wells were washed three times with a washing solution and the serum (4-fold dilution) from 29 patients with cedar pollinosis and healthy subjects were each added to separate wells. The system was then reacted at 37° C. for 4 hours. After the sera were removed, the wells were washed three times with a washing solution then reacted with anti-human IgE antibody (made by Pharmacia Inc.) at room temperature overnight. After washing three times with a washing solution, a substrate solution containing 0.1 mM 4-methylumbelliferyl-β-D-galacto-pyranoside/0.01 M phosphate buffer (pH 7.0), 0.1 M NaCl, 1 mM MgCl₂, 0.1% NaN₃ and 0.1% BSA was added, and the solution was incubated at 37° C. for 2 hours. A solution of 0.1 M glycine/NaOH (pH 10.3) was added to the wells to terminate the reaction. Fluorescent intensity was measured using a fluorophotometer (Labsystems). For positive control to each multi-epitope peptide, biotin-labeled rabbit anti-d epitope IgG and peroxidase-labeled streptoavidin (made by Pierce Inc.) were reacted.

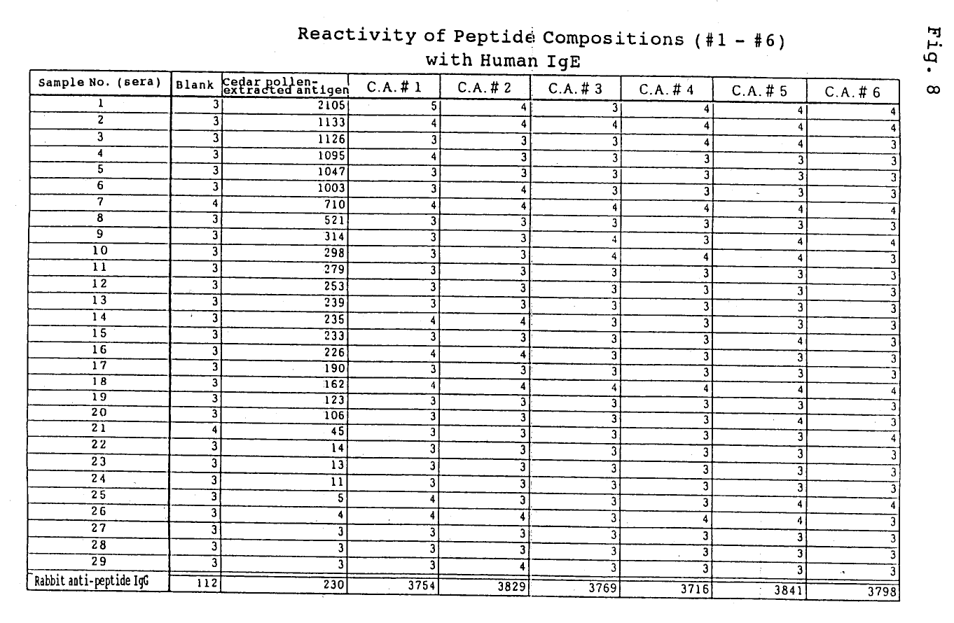

As a result, all sera from the 29 human subjects exhibited a fluorescent intensity of 3 to 5 to all of the six multi-epitope peptides (C.A.#1 through #6) (blank: 3 or 4). In contrast, when the antigen Cry j 1 extracted and purified from cedar pollen was used, a fluorescent intensity of 1,000 or more was noted in six subjects, 100 or more in 14 subjects, 10 or more in four subjects and nine or less in five subjects. In contrast, rabbit anti-d epitope peptide IgG exhibited a fluorescent intensity of 3,000 or more in response to the six consensus allergens (blank: 112; 230 to Cry j 1 allergen). These results reveal that the order of joining each epitope site in the multi-epitope peptide does not affect the reactivity with human IgE antibody (FIG. 8).

### Example 8

Recognizing the T Cell Epitopes in the Multi-Epitope Peptide

The antigenic peptide constituting the multi-epitope peptide C.A.#4 obtained in Example 6 was examined to determine if the antigenic peptide actually functions as a T cell epitope.

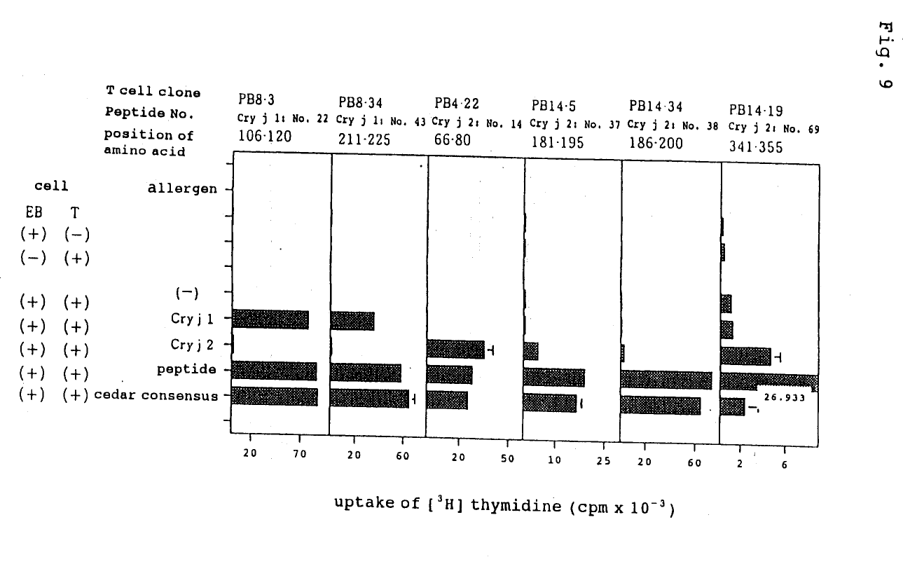

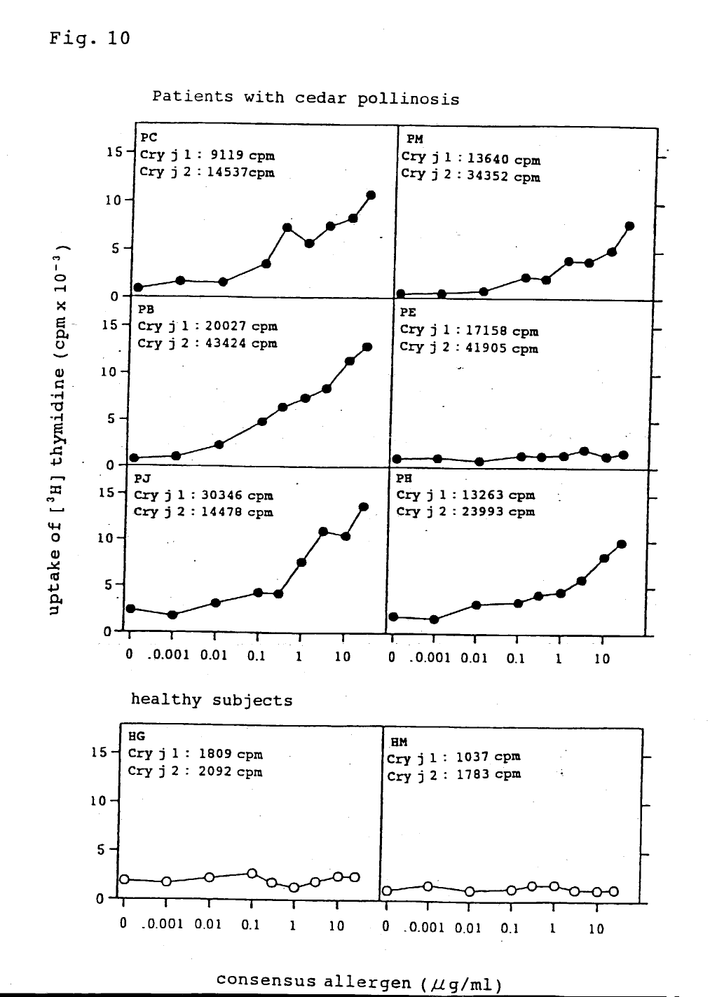

On a 96-well micro culture plate, a mixture of 5×10⁴ cells of the autologous B cell line previously treated with mitomycin C and 2×10⁴ cells of the T cell clone was incubated for 2 days in 0.2 ml 15% serum-supplemented RPMI-1640 medium, together with, as an antigen, either 50 μg/ml of Cry j 1 and 2 μg/ml of Cry j 2, each antigenic peptide constituting the multi-epitope peptide C.A.#4 or 10 μg/ml C.A.#4 multi-epitope peptide produced by gene expression. After 0.5 μCi [³H] thymidine was added to the medium, incubation was continued for a further 16 hours. After the cells were harvested on a glass filter using-a cell harvester, the level of [³H] thymidine taken up into the cells was determined using a liquid scintillation counter. The results are shown in FIG. 9.

T cell clone PB8-3 that recognizes Cry j 1 p106-120, T cell clone PB8-34 that recognizes Cry j 1 p211-225, T cell clone PB4-22 that recognizes-Cry j 2 p66-80, T cell clone PB14-5 that recognizes Cry j 2 p181-195, and T cell clone PB14-3 that recognizes Cry j 2 p186-200, all react well with the antigenic peptide. When the multi-epitope peptide was used, the T cell clones are responsive to proliferation at a level comparable to that of each of the peptides. The proliferation response of T cell clone PB14-19 that recognizes Cry j 2 p341-355 to the multi-epitope peptide stimulation was somewhat weak.

Those results indicate that the antigenic peptides contained in the multi-epitope peptide function well as the epitopes and retain the T cell activating ability.

### Example 9

Proliferation Response of the Peripheral Lymphocytes from Patients with Cedar Pollinosis Induced by Multi-Epitope Peptides

Since the multi-epitope peptide contains T cell epitope sites, it is necessary to induce proliferation response to peripheral lymphocytes upon applying peptide-based immunotherapy. The inventors thus examined if proliferation response is observed by stimulating peripheral lymphocytes with the multi-epitope peptide.

Peripheral lymphocytes derived from the patients with cedar pollinosis or from healthy subjects were suspended in RPMI-1640 culture medium supplemented with 10% human sera. The suspension was distributed in each well of a 96-well culture plate with a round bottom in a concentration of 2.5×10⁵ cells/200 μl. The multi-epitope peptide represented by SEQ NO: 1, either Cry j 1 or Cry j 2, was added to each well to a final concentration of 0.001 to 20 μg/ml of the multi-epitope peptide, 50 μg/ml of Cry j 1 or 2 μg/ml of Cry j 2. The platewas incubated for 6 days. After 0.5 μCi [³H] thymidine was added to the medium, incubation was continued for a further 16 hours. After the cells were harvested on a glass filter using a cell harvester, the level of [³H] thymidine taken up into the cells was determined using a liquid scintillation counter.

The peripheral lymphocytes from five out of the six patients showed proliferation response to the multi-epitope peptide. The peripheral lymphocytes from one patient and two healthy subjects showed no proliferation response (FIG. 10).

The proliferation response of peripheral lymphocytes began to occur with stimulation of 0.1 μg/ml of the multi-epitope peptide and increased dose-dependently. Based on the results, the concentration of the multi-epitope peptide required for inducing T cell proliferation response in vitro is at least 10 μg/ml.

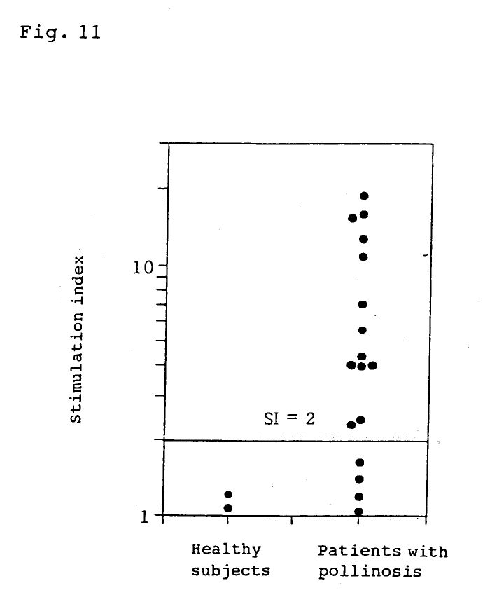

Peripheral lymphocytes from 17 patients with cedar pollinosis and two healthy subjects were stimulated by 10 μg/ml of the multi-epitope peptide to evaluate T cell response. No response to T cell proliferation was observed with the peripheral lymphocytes from the healthy subjects. In the 17 patients, a maximum [³H] thymidine uptake of 9,652 cpm was observed. When [³H] thymidine uptake of peripheral lymphocytes without antigen stimulation is regarded as 1, the uptake of [³H] thymidine by peripheral lymphocytes in the presence of an antigen is expressed by a stimulation index (SI). The results are shown in FIG. 11. Upon identification of T cell epitopes, SI>2 is regarded to be positive. Similarly, SI>2 is judged to be proliferation responsive to the peptide. Under this criterion, the proliferation response was noted in 13 out of the 17 patients (76.5%). From the results, the peptide-based immunotherapy is effective when administered to 76.5% of the cedar pollinosis patients.

When patients with cedar pollinosis are subjected to the peptide-based immunotherapy using the multi-epitope peptide of the present invention, the proliferation response capability of peripheral lymphocytes from the patients to the multi-epitope peptide can be tested in advance so that the patients responsive to proliferation can be selected. Such a test enables determining if the peptide-based immunotherapy using the multi-epitope peptide is applicable to the individual patient. Therapeutic effects can also be predicted to a certain extent, based on the level of proliferation response.

### Example 10

Inducing Immune Tolerance by Administering Cedar Pollen Allergen to Mice

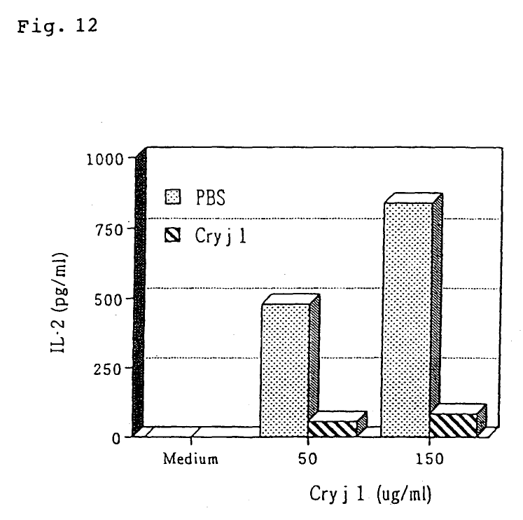

The detailed mechanism in hyposensitization therapy by which cedar pollen allergen is administered for the treatment is yet unknown. To clarify this mechanism, tests were conducted using mice. Cedar pollen allergen Cry j 1 was subcutaneously administered twice to five CBF1 female mice at intervals of 5 days in a dose of 300 μg/mouse. For control, the same dose of PBS was subcutaneously given to five other female mice. Five days later, the animals were sensitized by subcutaneous injection of 10 μg Cry j 1 together with Alum adjuvant. Ten days later, the lymphocytes were isolated to pool them as the lymphocytes from the control group and as the lymphocytes from the Cry j 1-administered mice. Cry j 1 was added to the pooled lymphocytes in doses of 0, 50 and 150 μg/ml. Incubation was performed for 3 days to collect the culture supernatant. IL-2 contained in the supernatant was measured with a device manufactured by Endogen Inc. The results are shown in FIG. 12. In the control (PBS-administered) mouse group, IL-2 production increased as the concentration of Cry j 1 increased from 0 to 50 and 150 μg/ml. In contrast, in the Cry j 1-administered mouse group, IL-2 production was obviously reduced, as compared to the control group, indicating that immune tolerance was acquired by administration of the cedar pollen allergen. The results verify that currently implemented hyposensitization therapy using the cedar pollen allergen is efficacious.

### Example 11

Identifying T Cell Epitopes in CBF1 Mice

Eight-week-old male CBF1 mice were boosted (i.p.) three times with 10 μg of recombinant Cry j 2 (rCry j 2) at intervals of two weeks together with an adjuvant (Imject Alum, manufactured by Pierce Inc.). One week after the final booster, the spleen cells were collected from three mice and mixed together. The spleen cells (5×10⁶ cells) were cultured together with each of the 74 kinds of Cry j 2 overlapping peptides (0.115 μM) consisting of 15 residues in 0.2 ml RPMI medium (supplemented with 10% FCS, 2 mM L-glutamine, 50 U/ml penicillin and 50 μg/ml streptomycin) in each well of a 96-well plate (manufactured by Falcon Inc.). For control, the reactivities with PBS, 50 μg/ml of Cry j 1, and 0.3 μg/ml of rCry j 2 were also observed. Each reagent was distributed in three wells and incubated at 37° C. for 3 days in 5% CO₂. For the last 6 hours, pulse labeling was performed with 0.5 μCi/well of [³H] thymidine. The cells were collected on a glass filter using a cell harvester (Inoteck, Bertold Japan Co., Ltd.). After drying, the level of [³H] thymidine taken up into the cells was determined using a liquid scintillation counter (TRI-CARB 4530,Packard Japan KK).

CBF1 mice immunized with rCry j 2 showed a strong reactivity with its antigen rCry j 2 but did not react with Cry j 1 which is another major cedar pollen allergen. This proved that the reaction is antigen-specific. Among the 74 overlapping peptides tested, CBF1 mice immunized with rCry j 2 showed marked response to Peptide Nos. 14 and 48 as shown in FIG. 2. These results indicate that Peptide Nos. 14 and 48 participate in antigen presentation as the major T cell epitopes in CBF1 mice. Peptides No. 14 and 48 are also known to be major T cell epitope peptides in humans. Therefore, CBF1 mice can be a useful animal model for judging the effectiveness of peptides used for the peptide-based immunotherapy against cedar pollen.

### Example 12

Immune Response of Antigenic Peptide No. 14 in Vivo

A solution of Peptide No. 14 (3 mg) in physiological saline was subcutaneously injected into each 8-week-old male CBF1 mouse (8 animals/group) twice, once and then again after a 5-day interval. For control, an equal volume (100 μl) of physiological saline was given to the control group in the same manner. On Day 5 after the second administration of the peptide, all mice were sensitized by subcutaneous injection with 50 μg/mouse of rCry j 2, together with Imject Alum. One week after the sensitization, the spleen cells were collected from each mouse. The spleen cells (5×10⁶ cells) were cultured together with 3 μg/ml of rCry j 2 in 0.2 ml RPMI medium (supplemented with 10% FCS, 2 mM L-glutamine, 50 U/ml penicillin and 50 μg/ml streptomycin) in each well of a 96-well plate (manufactured by Falcon Inc.). An incubation was also performed under the same conditions without rCry j 2 for comparison. T cell proliferation was determined in the same manner as in Example 1 using [³H] thymidine. Cytokine was determined using the culture supernatant obtained by stimulating the three peptide-administered groups (0.3, 1.3, and 10 μg/ml) and the control group with 0.3 μg/ml of Cry j 2 in vitro.

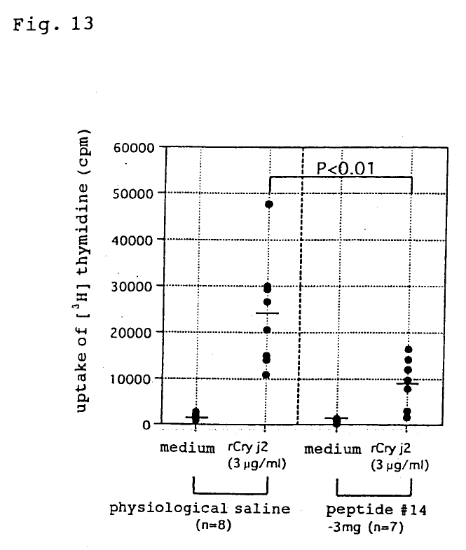

When CBF1 mice were previously subcutaneously administered Peptide No. 14, T cell immune response to the subsequent antigen stimulation by rCry j 2 was suppressed significantly (p<0.01), as compared to the physiological saline group (FIG. 13). The peptide-administered group showed a significant decrease in IL-2 production as compared to the control group. These results reveal that in the mouse model system, Peptide No. 14 exhibits the preventive effect for cedar pollinosis in the peptide-based immunotherapy.

### Example 13

Immune Response of Antigenic Peptide No. 48 in Vivo

A solution of Peptide No. 48 (3 mg) in physiological saline was subcutaneously injected into each 6-week-old male CBF1 mouse twice at intervals of 5 days. For control, an equal volume (200 μl) of physiological saline was given in the same manner. There were eight animals each in the peptide-administered group and in the control group. On Day 5 after the second administration of the peptide, all mice were sensitized by subcutaneous injection with 50 μg/mouse of rCry j 2 mixed with an adjuvant (Imject Alum). One week after the sensitization, the spleen cells were collected from each mouse. The spleen cells (5×10⁶ cells) were cultured together with 3 μg/ml of rCry j 2 in 0.2 ml RPMI medium (supplemented with 10% FCS, 2 mM L-glutamine, 50 U/ml penicillin, and 50 μg/ml streptomycin) in each well of a 96-well plate (manufactured by Falcon Inc.). An incubation was also performed under the same conditions without rCry j 2 for comparison. T cell proliferation was determined in the same manner as in Example 1 using [³H] thymidine.

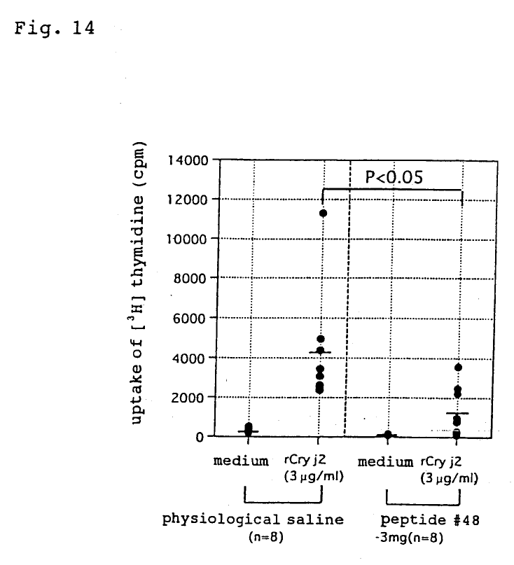

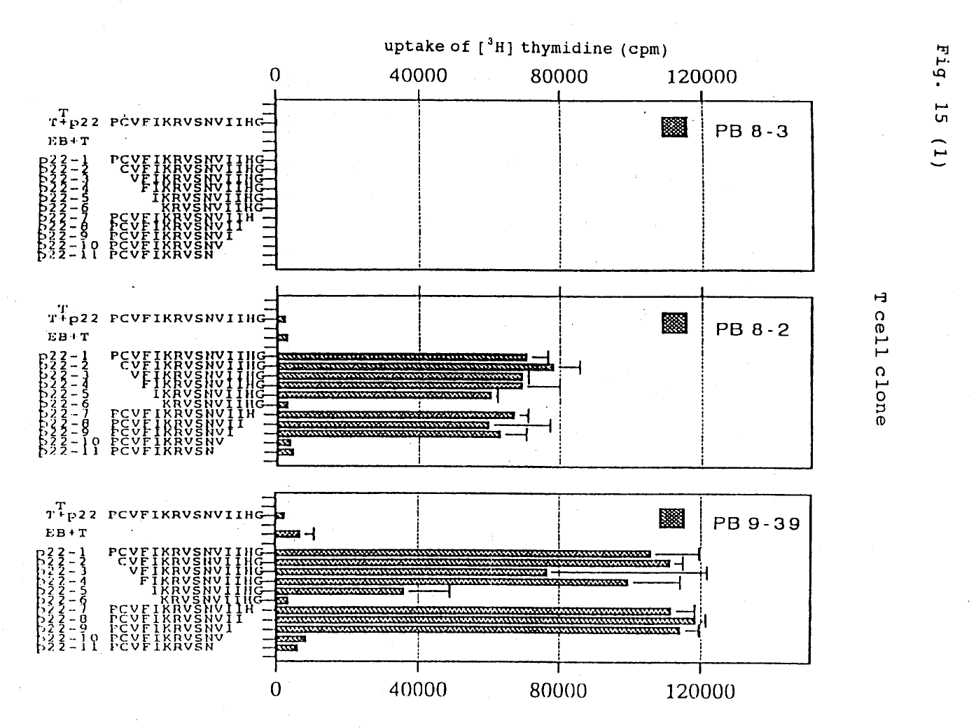

When CBF1 mice were previously subcutaneously administered Peptide No. 48, T cell immune response to the subsequent antigen stimulation by rCry j 2 was suppressed significantly (p<0.05), as compared to the physiological saline-administered group. This result indicates that in the mouse model system, Peptide No. 48 exhibits the preventive effect for cedar pollinosis in peptide-based immunotherapy (FIG. 14).

The experimental results described above reveal that the conventionally implemented hyposensitization therapy in humans using the cedar pollen extract works on the mechanism mediated by the T cell epitope.

### Example 14

Determination of Core Sequence

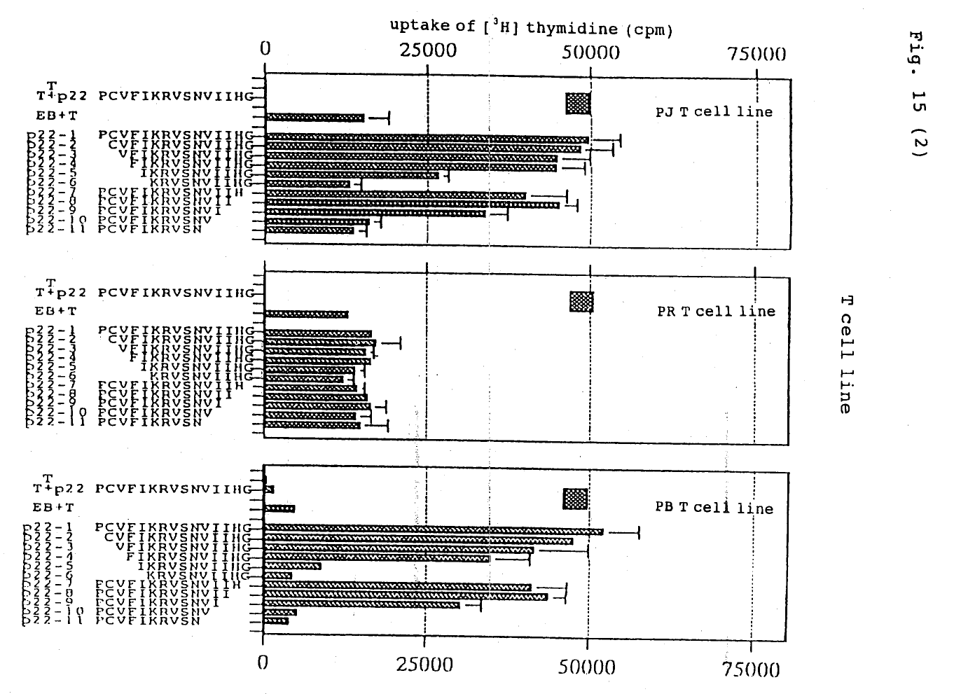

To determine the minimum amino acid sequence (core) of the Cry j 1 peptide No. 22 (p106-120) necessary for the T cell line and T cell clone proliferation response, one amino acid residue each was deleted from the N and C terminals of this peptide as shown in FIG. 15 to prepare eleven peptides, i.e., p107-120 (p22-2), P108-120 (p22-3), p109-120 (p22-4), p110-120 (p22-5), p111-120 (p22-6), p106-119 (p22-7), p106-118 (p22-8), p106-117 (p22-9), p106-116 (p22-10), and p106-115 (p22-11) using a peptide synthesizer (PSSM-8, manufactured by Shimadzu Seisakusho Ltd.). The T cell lines (PJ, PR, PB) derived from three patients with cedar pollinosis and which react with Cry j 1 Peptide No. 22, p106-120, and the T cell clones (PB 8-3, PB 8-2, PB 9-39) from one of the patients were examined for the reactivity with these 11 peptides in the same manner as in Examples 1 and 2. Two T cell lines (PJ, PB) and two T cell clones (PB 8-2, PB 9-39) recognized p106-120 (p22-1) and proliferated, but one T cell line and T cell clone did not show any proliferation response (FIG. 15). The results reveal that the p106-120 core sequence consists of nine residues of “FIKRVSNVI” (the nine residues are designated as Cry j 1 #22 core).

### Example 15

Multi-Epitope Peptide Containing T Cell Epitopes Derived from Cedar Pollen and Hinoki Pollen Allergens

Two peptides (Cha o 1 #8-Cry j 1 #22 core, Cha o 1 #32-Cry j 1 #22 core) were synthesized by joining Peptide No. 8 (p71-90: IFSKNLNIKLNMPLYIAGNK), which is a T cell epitope of hinoki pollen allergen Cha o 1 (Japanese Patent Application No. Hei 8-153527), or Peptide No. 32 (p311-330: SSGKNEGTNIYNNNEAFKVE) to Cry j 1 #22 core sequence “FIKRVSNVI” obtained in Example 14 using a peptide synthesizer (PSSM-8, Shimadzu Seisakusho Ltd.). An RR sequence was inserted between Cha o 1 #8 and Cry j 1 #22 core and between Cha o 1 #32 and Cry j 1 #22 core, that is, Cha o 1 #8-Cry j 1 #22 core (SEQ NO: 4) and Cha o 1 #32-Cry j 1 #22 core (SEQ NO: 5).

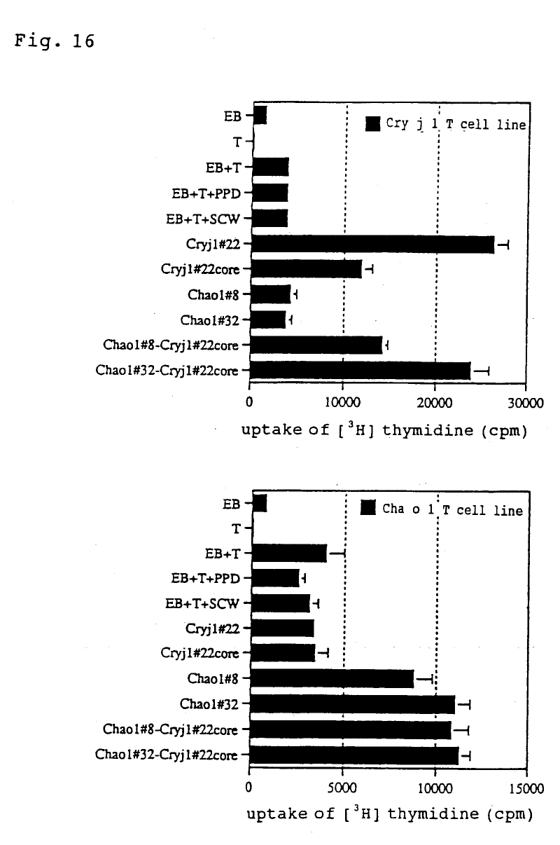

A Cry j 1-specific T cell line and a Cha o 1-specific T cell line were prepared from the patients with cedar pollinosis and hinoki pollinosis, respectively. The Cry j 1-specific T cell line and Cha o 1-specific T cell line react with neither the tubercle bacillus antigen (PPD) nor the hemolytic streptococcus cell wall (SCW) antigen. The Cry j 1-specific T cell line reacts with Cry j 1 #22 or Cry j 1 #22 core but does not react with Cha o 6 1 #8 or with Cha o 1 #32. The Cha o 1-specific T dell line reacts with Cha o 1 # 8 and #32 but does not react with Cry j 1 #22 or Cry j 1 #22 core (FIG. 16). However, these T cell lines all react with the multi-epitope peptide of SEQ NO: 4 and with the multi-epitope peptide of SEQ NO: 5. These results reveal that the multi-epitope peptides prepared by joining T cell epitopes derived from cedar pollen and hinoki pollen allergens are effective for peptide-based immunotherapy of patients with cedar pollinosis and with hinoki pollinosis.

### Example 16

The Proliferation Response and the Cytokine Production which Result from Addition of the Peptides

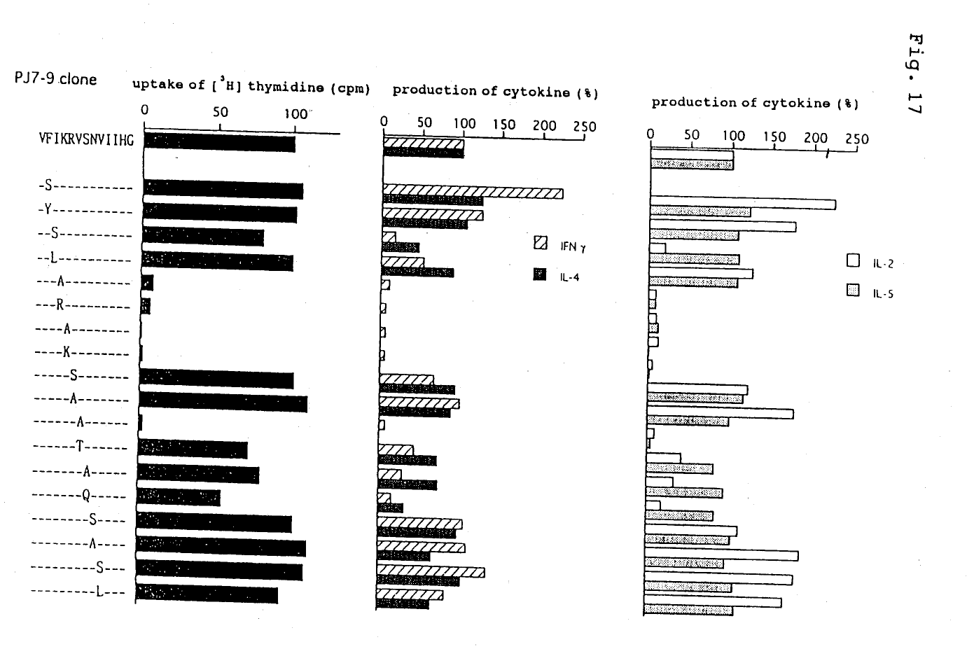

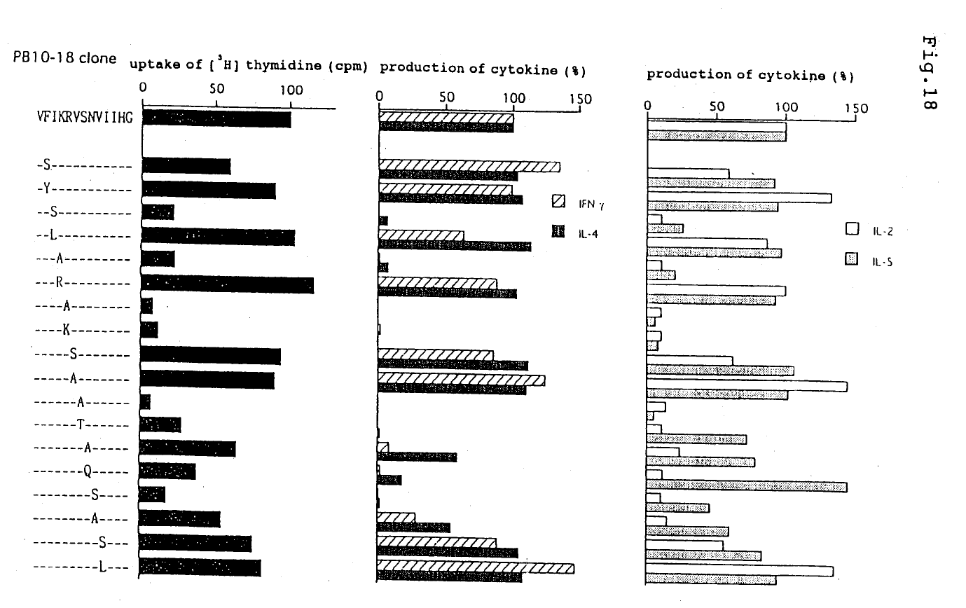

Two clones, PJ7-9 and PB10-18, were employed to see if the activity of T cells can be altered by substituting the amino acids of T cell epitope peptide of the Cry j 1 #22 core. T cell clones PJ 7-9 and PB10-12 which react with Cry j 1 Peptide No. 22 p106-120 are restricted by DRB5*0101 and recognize the nine residues of the Cry j 1 #22 core. Each of the nine amino acid residues in the peptide p108-120 (VFIKRVSNVIIHG) of 13 residues including the nine residues were substituted with an homologous amino acid and a non-homologous amino acid to produce analog peptides (FIGS. 17 and 18). The reactivity of T cell clones PJ 7-9 and PB 10-18 with these analog peptides was examined in terms of the uptake of [³H] thymidine. The concentration of cytokine in the reaction solution was measured using a cytokine assay kit manufactured by R & D Systems. The results are shown in FIGS. 17 and 18. The production of IFN-γ, IL-4, IL-2 , and IL-5 in the supernatant and the uptake of [³H] thymidine obtained by reacting the peptide of 13 residues with no amino acid substitution were regarded as 100%. In the PJ7-9 clone, the uptake of [³H] thymidine and cytokine production were both markedly suppressed by substituting amino acid Nos. 3, 4 and 6 in Cry j 1 #22 core,“FIKRVSNVI,” namely, “K,” “R,” and “S,” with both homologous and non-homologous amino acids or with only non-homologous amino acids (FIG. 17). Accordingly, the amino acids located in these positions are considered to be important for forming the complex of HLA and T cell receptor molecules via the peptide. Even though the first amino acid (F) is substituted with its homologous amino acid Y, no change is noted in the uptake of [³H] thymidine and production of IL-4 and IL-5, but substitution with a non-homologous amino acid “S” results in a marked increase in IFN-γ and IL-2 production, even though no change is observed in the uptake of [³H] thymidine. For the PB 10-18 clone, the uptake of [³H] thymidine is suppressed by substitution of amino acid No. 1, 2, 3, 4, 6, 7, or 8 of Cry j 1 #22 core. The amino acids located in these positions are considered to be important for forming the complex of HLA and T cell receptor molecules via the peptide. It is further observed that the IL-2 production is suppressed by substitution of amino acid No. 6, 7, or 8, as compared to IL-5 production (FIG. 18). These results reveal that “SIKRVSNVI” obtained by substituting the first amino acid F with S in Cry j 1 #22 core increases the production of IFN-γ and is thus effective as a therapeutic agent for allergy.

Industrial Applicability

The multi-epitope peptide of the present invention contains T cell epitopes derived from distinct allergen molecules. It contains the peptide presented on the HLA class II molecule encoded by the gene that frequently appears in the population of patients with allergy. It further contains several peptides presented on the HLA class II molecules in different loci (DR, DQ, DP). A peptide-based immunotherapy for effectively treating a wider range of patients could be realized using a multi-epitope peptide of a minimum length.

When patients with allergy are subjected to the peptide-based immunotherapy using the multi-epitope peptide of the present invention, the proliferation response of peripheral lymphocytes from the patients to the peptide can be tested prior to the therapy, to thereby select patients who produce the proliferation response. This test enables judging if the peptide-based immunotherapy with the multi-epitope peptide applies to the patients. The therapeutic effect is also predictable to a certain extent based on the level of the proliferation response.

We claim:

1. A peptide-based immunotherapeutic agent for cedar pollinosis comprising an effective amount of a polypeptide, wherein said polypeptide:
(a) is a polypeptide in which (i) at least one T-cell epitope peptide comprising an amino acid sequence selected from the group consisting of SEQ ID NO: 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, and 83 linearly bound to (ii) a T-cell epitope peptide comprising an amino acid sequence selected from the group consisting of SEQ ID NO: 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129,,130, 131, 132, 133, 134, 135, 136, 137, 138, 139, 140, 141, 142, 143, 144, 145, 146, 147, 148, 149, 150, 151, 152, 153, 154, 155, 156, and 157;
(b) does not substantively bind to cedar pollen allergen-specific IgE antibody in serum of cedar pollinosis patients,
(c) is capable of proliferating in vitro human T cell clones specific to the T cell epitope peptide within the peptides of (a),
(d) is capable of proliferating in vitro peripheral blood lymphocytes of a cedar pollinosis patient, and
(e) has no cysteine residues.

2. The peptide-based immunotherapeutic agent for cedar pollinosis according to claim 1, wherein said polypeptide comprises:
(a) a first T-cell epitope peptide consisting of an amino acid sequence selected from the group consisting of SEQ ID NO: 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, and 83 linearly bound to a second T-cell epitope peptide consisting of an amino acid sequence selected from the group consisting of SEQ ID NO: 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129,,130, 131, 132, 133, 134, 135, 136, 137, 138, 139, 140, 141, 142, 143, 144, 145, 146, 147, 148, 149, 150, 151, 152, 153, 154, 155, 156, and 157.

3. A peptide-based immunotherapeutic agent for cedar pollinosis comprising an effective amount of a polypeptide, wherein said polypeptide comprises:
(a) a first T-cell epitope peptide consisting of an amino acid sequence selected from the group consisting of SEQ ID NO: 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, and 83 linearly bound to a second T-cell epitope peptide consisting of an amino acid sequence selected from the group consisting of SEQ ID NO: 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129,,130, 131, 132, 133, 134, 135, 136, 137, 138, 139, 140, 141, 142, 143, 144, 145, 146, 147, 148, 149, 150, 151, 152, 153, 154, 155, 156, and 157.

4. The peptide-based immunotherapeutic agent according to claim 3 wherein said agent does not substantively bind to cedar pollen allergen-specific IgE antibody in serum of cedar pollinosis patients.

5. The peptide-based immunotherapeutic agent according to claim 4, wherein said agent is capable of proliferating in vitro human T cell clones specific to the T cell epitope peptides.

6. The peptide-based immunotherapeutic agent according to claim 5, wherein said agent is capable of proliferating in vitro peripheral blood lymphocytes of a cedar pollinosis patient.

7. The peptide-based immunotherapeutic agent according to claim 6, wherein said agent has no cysteine residues.

8. The peptide-based immunotherapeutic agent according to claim 1, wherein said agent further comprises a cleavage site between said T-cell epitope peptides.

9. The peptide-based immunotherapeutic agent according to claim 2, wherein said agent further comprises a cleavage site between said T-cell epitope peptides.

10. The peptide-based immunotherapeutic agent according to claim 3, wherein said agent further comprises a cleavage site between said T-cell epitope peptides.

11. A composition comprising a carrier and a peptide-based immunotherapeutic agent for cedar pollinosis comprising an effective amount of a polypeptide, wherein said polypeptide:
(a) is a polypeptide in which (i) at least one T-cell epitope peptide comprising an amino acid sequence selected from the group consisting of SEQ ID NO: 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, and 83 linearly bound to (ii) a T-cell epitope peptide comprising an amino acid sequence selected from the group consisting of SEQ ID NO: 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129,,130, 131, 132, 133, 134, 135, 136, 137, 138, 139, 140, 141, 142, 143, 144, 145, 146, 147, 148, 149, 150, 151, 152, 153, 154, 155, 156, and 157;
(b) does not substantively bind to cedar pollen allergen-specific IgE antibody in serum of cedar pollinosis patients,
(c) is capable of proliferating in vitro human T cell clones specific to the T cell epitope peptide within the peptides of (a),
(d) is capable of proliferating in vitro peripheral blood lymphocytes of a cedar pollinosis patient, and
(e) has no cysteine residues.

12. The composition according to claim 11, wherein said polypeptide comprises:
(a) a first T-cell epitope peptide consisting of an amino acid sequence selected from the group consisting of SEQ ID NO: 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, and 83 linearly bound to a second T-cell epitope peptide consisting of an amino acid sequence selected from the group consisting of SEQ ID NO: 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129,,130, 131, 132, 133, 134, 135, 136, 137, 138, 139, 140, 141, 142, 143, 144, 145, 146, 147, 148, 149, 150, 151, 152, 153, 154, 155, 156, and 157.

13. The composition according to claim 11, wherein said agent further comprises a cleavage site between said T-cell epitope peptides.

14. The composition according to claim 12, wherein said polypeptide further comprises a cleavage site between said T-cell epitope peptides.

15. A method of treating cedar pollinosis comprising the administration of an effective amount of a composition comprising a carrier and a peptide-based immunotherapeutic agent for cedar pollinosis comprising an effective amount of a polypeptide, wherein said polypeptide:
(a) is a polypeptide in which (i) at least one T-cell epitope peptide comprising an amino acid sequence selected from the group consisting of SEQ ID NO: 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, and 83 linearly bound to (ii) a T-cell epitope peptide comprising an amino acid sequence selected from the group consisting of SEQ ID NO: 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129,,130, 131, 132, 133, 134, 135, 136, 137, 138, 139, 140, 141, 142, 143, 144, 145, 146, 147, 148, 149, 150, 151, 152, 153, 154, 155, 156, and 157;
(b) does not substantively bind to cedar pollen allergen-specific IgE antibody in serum of cedar pollinosis patients,
(c) is capable of proliferating in vitro human T cell clones specific to the T cell epitope peptide within the peptides of (a),
(d) is capable of proliferating in vitro peripheral blood lymphocytes of a cedar pollinosis patient, and
(e) has no cysteine residues.

16. The method according to claim 15, wherein said polypeptide comprises:
(a) a first T-cell epitope peptide consisting of an amino acid sequence selected from the group consisting of SEQ ID NO: 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, and 83 linearly bound to a second T-cell epitope peptide consisting of an amino acid sequence selected from the group consisting of SEQ ID NO: 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129,,130, 131, 132, 133, 134, 135, 136, 137, 138, 139, 140, 141, 142, 143, 144, 145, 146, 147, 148, 149, 150, 151, 152, 153, 154, 155, 156, and 157.

17. The method according to claim 16, wherein said agent further comprises a cleavage site between said T-cell epitope peptides.

18. The method according to claim 17, wherein said polypeptide further comprises a cleavage site between said T-cell epitope peptide.
